(19) United States
(12) Patent Application Publication
(10) Pub. No.: US 2014/0314723 A1
(43) Pub. Date: Oct. 23, 2014

(54) ENDOTHELIAL CELL CULTURE

(71) Applicant: National University of Singapore, Singapore (SG)

(72) Inventors: King Fai Evelyn Yim, Singapore (SG); Kim Kiat Teo, Singapore (SG); Stephanie Koo, Singapore (SG)

(73) Assignee: National University of Singapore, Singapore (SG)

(21) Appl. No.: 14/255,032

(22) Filed: Apr. 17, 2014

Related U.S. Application Data
(60) Provisional application No. 61/812,826, filed on Apr. 17, 2013.

(51) Int. Cl. C12N 5/079; A61K 35/44; G01N 33/50; C12N 5/00

(52) U.S. Cl. CPC C12N 5/0621; C12N 5/0075; A61K 35/44; G01N 33/5064
USPC 424/93.7; 435/299.1; 435/377; 435/366; 435/29; 506/10; 435/7.21; 435/6.13; 435/6.12; 435/19

(57) ABSTRACT

The disclosure relates to cell culture vessels comprising fabricated cell culture surfaces providing means for efficient corneal endothelial cell growth and the use of the cultured cells in the repair of corneal tissue damage.

This utility patent application claims the benefit of priority in U.S. Provisional Patent Application Ser. No. 61/812,826 filed on Apr. 17, 2013, the entirety of the disclosure of which is incorporated herein by reference.

### TECHNICAL FIELD

The disclosure relates to cell culture vessels comprising fabricated cell culture surfaces providing means for efficient growth of mammalian corneal endothelial cells or mammalian corneal endothelial stem cells, in particular primary corneal endothelial cells and corneal endothelial stem cells; a method of growing mammalian corneal endothelial cells or mammalian corneal endothelial stem cells on said cell culture surfaces in vitro; use of the cultured endothelial cells or corneal endothelial stem cells in the repair of damaged or diseased corneal tissue; and kits enabling the growth of corneal endothelium.

### BACKGROUND

The ability to see is dependent on the actions of several structures in and around the eye.

When one focuses on an object, light rays are reflected from the object to the cornea. The light rays are bent, refracted and focused by the cornea, lens, and vitreous within the eye ball. The lens functions to ensure that light is focused on the retina. The retina then converts light into electrical impulses which are transmitted through the optic nerve to the brain where the image is perceived. The cornea is a highly organised structure being composed of different layers of cells comprising the anterior epithelium, Bowman's layer, corneal stroma, Descemet's membrane and corneal endothelium.

The corneal endothelium is a monolayer of cells separating the corneal stroma from the anterior chamber. This layer controls corneal hydration and is essential for the thickness and transparency of the cornea by removing excess fluid from the stroma to prevent distortion and opaqueness in the cornea. Unlike corneal endothelial cells of other species, mature human corneal endothelial cells do not proliferate in vivo to replace those lost due to cell injury or death [2]. The area deficit caused by the death of corneal endothelial cells is instead compensated by cell enlargement and migration. When excessive endothelial cell loss occurs the endothelium loses its function causing corneal edema in the stroma and decreased corneal clarity which eventually leads to loss of vision [3,4]. If the endothelial layer is destroyed then the only corrective therapy is corneal transplantation.

There are a large number of diseases and conditions which affect the function of the cornea requiring transplantation of corneal tissue from donors such as Fuchs' dystrophy, iridocorneal endothelial syndrome, keratoconus, lattice dystrophy, ocular herpes infections, trachoma or damage by chemical burns. Existing available surgical treatments include penetrating keratoplasty (full-thickness transplantation) and endothelial keratoplasty, by which the patient's endothelium is replaced with a transplanted disc of posterior stroma/Descemet's membrane/corneal endothelium or Descemet's membrane/corneal endothelium [6].

The reconstruction of corneal endothelium has been of great interest due to its potential as a tissue-engineered replacement [7,8] or even as a tool for in vitro toxicology testing [9]. In vitro ocular toxicity tests offer a number of advantages over conventional animal tests. They are more economical, faster, avoid ethical issues with animal models, and reduce inconsistencies among data generated from different species [9,10]. Monolayers and stratified layers of corneal epithelial cells have been commonly used for testing drugs, contact lens or other irritants [11, 12]. Recently, in vitro cultures of corneal endothelial cells have also been investigated as a means to test for toxic effects [13-15].

Several patent applications concern methods and compositions stimulating proliferation of human corneal endothelial cells. Patent application US20100003299 discloses methods and compositions for stimulating proliferation of human corneal endothelial cells by down regulation of certain cell-cell junctions. Others such as U.S. Pat. No. 6,548,059 or WO2005038015 describe specific culture compositions improving corneal endothelial cell proliferation. However, cells respond also to different synthetic topographic substrates and the physical properties of the adherent material [18]. Synthetic substrate topographies of nanoscale and submicron dimensions have been found to modulate human corneal epithelial cell (HCEpiC) behaviors such as morphology and cell orientation [19]. Tocce et al., (2010) analysed the impact of topography on corneal cells behaviour and proliferation and utilized well defined ridge and groove wave-like nanostructures to improve cell proliferation. Although the advantages in providing means and methods improving corneal endothelial cell proliferation are apparent, tissue grafts with characteristics found in healthy corneal endothelium suitable for long lasting transplants have not yet been obtained.

This disclosure relates to optimal conditions for the growth and proliferation of mammalian corneal endothelial cells or mammalian corneal endothelial stem cells that proliferate extremely well when seeded on fabricated cell culture surfaces which comprise raised pillars. Cell densities and functional characteristics mirror those found in healthy corneal endothelium making the cultured endothelia cells suitable for corneal repair of damaged corneal tissue.

### SUMMARY

According to an aspect of the invention there is provided a cell culture vessel for use in the proliferation and differentiation of mammalian corneal endothelial cells or mammalian corneal endothelial stem cells comprising a cell culture surface wherein said culture surface comprises a plurality of upwardly projecting pillars from a surface wherein the pillars are substantially spaced equidistant from each other and provides a culture surface to which said endothelial cells or endothelial stem cells have enhanced adherence and proliferate and differentiate to form a monolayer of functional endothelial cells.

“Cell culture vessel” is defined as any means suitable to contain a cell culture of endothelial cells or endothelial stem cells. Typically, an example of such a vessel is a petri dish or multiwell culture dishes or a well insert/cell culture substrate adapted by provision of a cell culture surface according to the invention. Multi-well culture dishes are micro-titre plates with formats such as 6, 12, 48, 96 and 384 wells and which are typically compatible with automated loading and robotic handling systems.

In an embodiment of the invention said cell culture surface is provided on a substrate adapted to fit within said cell culture vessel.

It will be apparent that the provision of a substrate comprising a cell culture surface according to the invention will facilitate sample handling and processing of cultivated endothelial cells or endothelial stem cells.

In an embodiment of the invention said cell culture vessel or substrate comprises thermoplastic or elastic polymers.

In an embodiment of the invention said thermoplastic polymers or elastic polymers are selected from the group consisting of: polymethylmethacrylate, polydimethylsiloxane, polysterene, polyester or polypropylene.

In an embodiment of the invention said polymer is polydimethylsiloxane.

In an embodiment of the invention said pillars have a substantially circular cross-section.

In an embodiment of the invention said pillars are between 100 nm and 1 μm in height +/−15%.

The height of said pillars may be selected from the group consisting of: 100 nm, 200 nm, 300 nm, 400 nm, 500 nm, 600 nm, 700 nm, 800 nm, 900 nm, 1 μm or 2 μm.

The height of said pillars may be selected from the group consisting of: 200 nm, 210 nm, 220 nm, 230 nm, 240 nm, 250 nm, 260 nm, 270 nm, 280 nm, 290 nm or 300 nm.

In an embodiment of the invention the height of said pillar is about 250 nm

In an alternative embodiment of the invention the height of said pillar is about 1 μm.

In an embodiment of the invention the spacing between said pillars is between about 100 nm and 300 nm +/−15%.

In an embodiment of the invention the spacing between said pillars is selected from the group consisting of: 100 nm, 125 nm, 150 nm, 175 nm, 200 nm, 225 nm, 250 nm, 275 nm or 300 nm.

In an alternative embodiment of the invention the spacing between said pillars is between about 1 μm and 6 μm +/−15%.

In an embodiment of the invention the spacing between said pillars is between 1 μm and 6 μm.

In an embodiment of the invention the spacing is about 250 nm.

In an embodiment of the invention said cell culture vessel or substrate is adapted by provision of one or more extracellular matrix proteins to facilitate endothelial cell growth and differentiation.

In an embodiment of the invention said extracellular matrix protein comprises laminin.

In an alternative embodiment of the invention said extracellular matrix protein comprises laminin and chondroitin sulphate.

In an alternative embodiment of the invention said extracellular matrix protein comprises fibronectin and collagen.

In an embodiment of the invention said cell culture vessel or substrate is seeded with a cell culture composition comprising mammalian corneal endothelial cells or mammalian corneal endothelial stem cells and including cell culture medium.

In an embodiment of the invention said cell culture medium is a stabilisation medium and comprises: human endothelial SFM, 5% v/v Fetal Bovine Serum and 1% antimycotic.

In an alternative embodiment of the invention said cell culture medium is a proliferation medium and comprises: Ham's F12/M199, 5% v/v fetal bovine serum, 241 g/ml ascorbic acid, a combination of insulin, transferrin and selenium, 1% antimycotic and 10 ng/ml bFGF.

In an embodiment of the invention said corneal endothelial cells or corneal endothelial stem cells are allogenic.

In an alternative embodiment of the invention said mammalian corneal endothelial cells or mammalian corneal endothelial stem cells are autologous.

In a further embodiment of the invention said mammalian corneal endothelial cells or mammalian corneal endothelial stem cells are isolated from a non-human primate.

In a further embodiment of the invention said mammalian corneal endothelial cells or mammalian corneal endothelial stem cells are isolated from a bovine species.

In a further embodiment of the invention said mammalian corneal endothelial cells or mammalian corneal endothelial stem cells are isolated from a human subject.

According to a further aspect of the invention there is provided a method for the culture of mammalian corneal endothelial cells or mammalian corneal endothelial stem cells comprising the steps:
i) providing a cell culture vessel or substrate according to the invention and seeding said vessel or substrate with mammalian corneal endothelial cells or mammalian corneal endothelial stem cells; and
ii) providing cell culture conditions to allow the proliferation and differentiation of said cells.

In a method of the invention said mammalian corneal endothelial cells or mammalian corneal endothelial stem cells are allogenic.

In an alternative method of the invention said mammalian corneal endothelial cells or mammalian corneal endothelial stem cells are autologous.

In an alternative method of the invention said mammalian corneal endothelial cells or mammalian corneal endothelial stem cells are isolated from a non-human species.

In a further method of the invention said non-human species is a non-human primate. In a further method of the invention said non-human species is a bovine species.

In a method of the invention said mammalian corneal endothelial cells or mammalian corneal endothelial stem cells are seeded at a concentration of at least 2,500 cells/cm².

In a method of the invention said mammalian corneal endothelial cells or mammalian corneal endothelial stem cells are seeded at a concentration of between 40,000 cells/cm² and 60,000 cells/cm².

In a method of the invention said mammalian corneal epithelial cells mammalian corneal endothelial stem cells are cultured in a stabilisation medium according to the invention.

In an alternative method of the invention said mammalian corneal epithelial cells mammalian corneal endothelial stem cells are cultured in a proliferation medium according to the invention.

According to a further aspect of the invention there is provided a therapeutic cell composition obtained or obtainable by the method according to the invention for use in the repair of damaged or diseased corneal tissue.

The cell composition according to the invention is administered in effective amounts. An “effective amount” is that amount of the cell composition that alone, or together with further doses, produces the desired response. In the case of treating corneal disease the desired response is inhibiting progression of disease or more preferably reversing disease. This may involve only slowing the progression of the disease temporarily, although more preferably, it involves halting the progression of the disease permanently. Such amounts will depend, of course, on the particular condition being treated, the severity of the condition, the individual patient parameters including age and physical condition, the duration of the treatment, the nature of concurrent therapy (if any) and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In an embodiment of the invention said corneal endothelial disease is selected from the group consisting of: Fuch's dystrophy, keratoconus, lattice dystrophy, map-dot finger print dystrophy, iridocorneal endothelial syndrome, iris nevus syndrome, Chandler's syndrome, essential iris atrophy, or Steven's Johnson syndrome.

In an alternative embodiment of the invention said disease is the result of infection.

Said infection may be caused by Chlamydia trachomatis.

Alternatively said infection may be caused by herpes simplex virus.

In an alternative embodiment of the invention said damaged corneal tissue is the result of a chemical burn.

According to a further aspect of the invention there is provided a method to screen for an agent wherein said agent affects the proliferation, differentiation or function of mammalian corneal endothelial cells or mammalian corneal endothelial stem cells comprising the steps of:
i) providing a cell culture vessel comprising mammalian corneal endothelial cells or mammalian corneal endothelial stem cells according to the invention;
ii) adding at least one agent to be tested; and
iii) monitoring the activity of the agent with respect to the proliferation, differentiation or function of said corneal endothelial cells or corneal endothelial stem cells.

In a method of the invention said screen is a high throughput screen wherein said agent[s] is contacted with a plurality of cell culture preparations.

In a method of the invention said cell culture vessel is adapted to co-operate with a fluorimetric plate reader.

In a method of the invention said screening method includes the steps of: collating the activity data in (iii) above; converting the collated data into a data analysable form; and optionally providing an output for the analysed data.

A number of methods are known which image and extract information concerning the spatial and temporal changes occurring in cells expressing, for example fluorescent proteins and other markers of gene expression, (see Taylor et al Am. Scientist 80: 322-335, 1992), which is incorporated by reference. Moreover, U.S. Pat. No. 5,989,835 and U.S. Ser. No. 09/031,271, both of which are incorporated by reference, disclose optical systems for determining the distribution or activity of fluorescent reporter molecules in cells for screening large numbers of agents for biological activity. The systems disclosed in the above patents also describe a computerised method for processing, storing and displaying the data generated.

The screening of large numbers of agents requires preparing arrays of cells for the handling of cells and the administration of agents. Assay devices, for example, include standard multi-well plates with formats such as 6, 12, 48, 96 and 384 wells which are typically used for compatibility with automated loading and robotic handling systems. Typically, high throughput screens use homogeneous mixtures of agents with an indicator compound which is either converted or modified resulting in the production of a signal. The signal is measured by suitable means (for example detection of fluorescence emission, optical density, or radioactivity) followed by integration of the signals from each well containing the cells, agent and indicator compound.

According to a further aspect of the invention there is provided a kit comprising a cell culture vessel according to the invention and optionally including cell culture medium for the culture of mammalian corneal endothelial cells or mammalian corneal endothelial stem cells.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

An embodiment of the invention will now be described by example only and with reference to the following figures:

FIG. 1: Scanning electron micrograph characterization of the micro and nanotopography fabricated by soft-lithography used in the studies. Starting from top left, micron sized topographies used included (A) Channels, (B) Gratings, (C) Concentric circles, (D) Round wells, (E) Round pillars, (F) 1 μm pillars, (G) 1 μm wells and nano sized topographies of (H) 250 nm pillars and (I) 250 nm wells. (Scale bars=20 μm unless specified);

FIG. 2: Screen for geometric patterns that can influence corneal endothelial cell functions. Immuno-fluorescent (IF) images of BCECs on micron scale (A) concentric circles, (B) parallel gratings, (C) pillars and (D) unpatterned PDMS control. BCECs were stained for actin (green), vinculin (red) and DAPI (blue). From the IF images, it can be observed that geometry changed cell shape and orientation. Focal adhesion (FA) indicated by vinculin is observed to elongate and co-localize with actin on the gratings in a parallel fashion. (FIG. 2(B), enlarged image of section showed in the insert.) Orientation of gratings is indicated by the vertical arrow. (Scale Bar=100 μm unless specified). The cell behavior and functions tested includes (E) proliferation in terms of cell densities (cells/mm2), (F) spreading (cell area) and (G) morphology (cell circularity) as healthy BCECs in vivo has a hexagonal and regular shape. *** indicates significant difference with p<0.005, ** p<0.01, * p<0.05 using one way ANOVA and Bonferroni post-test correction;

FIG. 3: Effect of topography on BCECs cell density. (A) Average cell density of BCECs cultured on a control unpatterned PDMS, 1 μm wells, 1 μm pillars and 250 nm wells six days after seeding. (B) Corresponding fluorescence images of BCECs on an unpatterned PDMS, 250 nm wells, 1 μm pillars and 1 μm wells six days after seeding. Cells were stained for their nucleus (blue) and F-actin filaments (green). (Scale bars=250 μm) A higher cell density was observed for cells seeded on the micro pillars as compared to the other substrate types indicated by both (B) fluorescence staining and (A) cell density (cells/mm2) quantification. Results are shown in mean±standard deviation. (C) Effect of topographical features on BCECs cellular morphology. Average cell circularity of BCECs cultured on unpatterned PDMS, 1 μm wells, 1 μm pillars and 250 nm wells six days after seeding. Circularity=4π×(area/perimeter2). A circularity value of 1.0 indicates a perfect circle. (D) BCECs seeded on 250 nm wells, 1 μm pillars, 1 μm wells and unpatterned control stained for F-actin (green) and DAPI (blue) after six days. (Scale bars=50 μm) BCECs cultured on the 250 nm wells were less circular and had extensions of actin filaments in random directions, assuming a more stellate morphology (white arrows) while on the other substrates, the BCECs adopted a rounded morphology. Quantification of BCECs cell circularity on 250 nm wells showed a significant difference compared to all other substrates, further supporting the observed cellular morphology in (D). (E) Average cell area of BCECs cultured on an unpatterned PDMS, micron sized (1 μm pillars and 1 μm wells) and nano-sized (250 nm wells and 250 nm pillars) topographies after six days. (F) BrdU expression of BCECs cultured on unpatterned PDMS, 1 μm wells, 1 μm pillars, 250 nm wells and 250 nm pillars six days after seeding. *** indicates significant difference with p<0.005, ** p<0.01, * p<0.05 using one way ANOVA and Bonferroni post-test correction;

FIG. 4: Immunofluorescence images of BCECs on various geometries such as micron and nano size pillars and wells that were previously identified to significantly influence BCECs cellular behavior. (A-D) showed BCECs stained for zonula occludens (ZO1, red) and DAPI (blue) after 3 days of culture while (E-I) were representative of BCECs on the various geometries after 6 days of culture. The formation of tight junctions in a BCEC monolayer is crucial for corneal endothelium function. In this study, it was shown that the use of nano sized pillars effected a stronger ZO1 expression, suggesting an improvement of BCECs functionality as indicated by the higher red intensity expressed by BCECs on 250 nm sized pillars after six days. Arrows indicate the presence of substantial ZO1 expression. (Scale bars=50 μm);

FIG. 5: (A) Cell area coefficient of variation (CV) of BCECs after six days of culture on the different topographies. The CV for BCECs cultured on both micron and nano sized wells were significantly different compared to control while the CV on 250 nm pillars was significantly lower when compared to all other samples. *** indicates significant difference with p<0.005, ** p<0.01, * p<0.05 using one way ANOVA and Bonferroni post-test correction. (B) Scanning electron micrographs of the BCEC monolayers on the respective topographies six days after seeding. Microvilli (bold arrows) formation was observed in the BCECs on all five different substrates. The microvilli, however, were more pronounced and dense on the 1 μm pillars, 1 μm wells and 250 nm pillars. Ruffled cell borders (thin arrows) could be observed predominantly on the cells cultured on the micro pillars, micro wells, and the flat substrate. (Scale bars=10 μm);

FIG. 6: (A) Confocal images of BCECs stained for Na+/K+ ATPase (Green) and DAPI (Blue) on the different topographies. Na+/K+ ATPase was substantially positively stained in all topographical samples although the unpatterned control showed low levels of Na+/K+ ATPase staining. (B) Gene expression analysis of BCECs functionality markers like Na+/K+ ATPase (ATP1A1), Aquaporin1 (AQP1), Voltage Dependant Anion Channel 3 (VDAC3) and collagen IV using reverse transcriptase PCR. GAPDH was used as an endogenous control. Notably, BCECs that were cultured on 250 nm pillars substrate showed an upregulation of ATP1A1, AQP1 and VDAC3 compared to control and the other topographies. The proper functioning of the Na+/K+ ATPase pump is the major function of the corneal endothelium. Our results suggests the improved functionality of BCECs under the influence of topography, more specifically nano sized pillars;

FIG. 7 is a diagrammatic representation of cell culture conditions for human endothelial cell line HCEC-B4G12 and primary human endothelial cells;

FIG. 8 illustrates the effect of TCPS topography on the proliferation of human primary corneal endothelial cell proliferation;

FIG. 9 illustrates the growth of human primary corneal endothelial cell growth and morphology in proliferation [P-media] and stabilisation media [S-media];

FIG. 10 illustrates Na⁺/K⁺-ATPase expression by human primary corneal endothelial cells grown in cell culture using various cell culture substrate topographies;

FIG. 11 illustrates ZO-I expression by human primary corneal endothelial cells grown in cell culture using various cell culture substrate topographies;

FIG. 12 illustrates the effect of cell culture substrate topography on the shape of human primary corneal endothelial cells; and

FIG. 13 illustrates the effect of cell culture substrate topography on high density human primary corneal endothelial cells marker expression.

### DETAILED DESCRIPTION

### Methods and Materials

Preparation of Polydimethylsiloxane (PDMS) Substrates

Soft lithography was used to fabricate polydimethylsiloxane (PDMS) substrates with micro- and nano-topographies as previously described [28]. Original patterned master molds were commercially purchased in silicon wafer format. Briefly poly(methyl methacrylate) (PMMA) (Microresist, PMMA, MW 35000 g/mol) was first spin-coated on a clean silicon substrate to form a thin PMMA film before the original master mold was placed on top of the spin-coated surface and the imprinting was carried out at 150° C. under a pressure of 60 bar for 10 min. Subsequently, the system was cooled before demolding the silicon master from the imprinted PMMA polymer layer. The PMMA mold is then subsequently used for soft lithography. For the initial geometry screening, an array of microscale patterns consisting of channels, gratings, concentric circles, round wells, round pillars, packed wells and packed pillars were used. Based on the observations from the initial screening, 1 μm pillars, 1 μm wells, 250 nm pillars and 250 nm wells were chosen to study the endothelial monolayer characterization. The master molds were cleaned using nitrogen gas and fluorinated with (tridecafluoro-1,1,2,2-tetrahydrooctyI)-1-trichlorosilane (Sigma Aldrich), before washing with 0.01% Triton X (BioRad). PDMS base and curing agent (Sylgard 184 Silicone Elastomer Kit, Dow Corning) was mixed using a 10:1 ratio and degassed in a dessicator for 30 minutes. The elastomer mixture was then poured onto the prepared masters, degassed and baked at 65° C. for at least 2 hours before the PDMS substrates were gently peeled off from the master molds.

The PDMS substrates were sputter coated with gold (JEOL JFC 1600 Fine Gold Coater) and examined using scanning electron microscopy (SEM, Quanta FEG 200 and JEOL JSM-5600LV Scanning Microscope) to verify the surface morphology and fidelity of the micro- and nano-topographies replication process. The dimensions of the replicated patterns on PDMS were verified with SEM and were expected to be within 20% of the intended dimensions of the mold design.

In preparation for cell seeding, all substrates were air plasma-treated with low RF power for 15 seconds (Harrick Scientific Corporation PDC-002), sterilized with 70% ethanol and UV irradiated, and pre-coated with 5 μg/ml of Laminin-1 from Engelbreth-Holm-Swarm murine sarcoma (basement membrane) at 37° C. (Invitrogen) overnight before BCECs were seeded at the relevant density.

### CORNEAL ENDOTHELIAL CELL CULTURE ON PDMS SUBSTRATES

### Bovine Corneal Endothelial Cells

Bovine Corneal Endothelial Cells (BCE C/D-1b, ATCC [29]) were cultured according to suppliers' specifications. Complete BCEC medium was made using Dulbecco's Modified Eagle's Medium (Sigma Aldrich) supplemented with 10% fetal bovine serum (Gibco, Invitrogen). BCECs were routinely cultured on standard tissue culture flask until a confluent monolayer was obtained. For all samples, BCECs were cultured on the air-plasma treated PDMS. For BCE monolayer analysis, BCECs were seeded at two different densities 60,000 cells/cm2 and 40,000 cells/cm2. Preliminary test was performed to determine these two densities where the BCEC monolayers would be formed on the 2nd and 5th day, one day prior to the analysis. The samples were analyzed on the 3rd and the 6th day, respectively. It was observed in an optimization study that the duration of cell-substrate interaction before confluence could affect cell response, therefore, two different seeding densities, which rendered 2 different culture periods, were studied. Culture medium was changed once every two days after seeding for all samples. The cells were suspended in 1 ml of culture medium and seeded on the prepared samples in 24 well plates and incubated at 37° C. and 5% CO2.

Human Corneal Endothelial Cells [B4G12]

Clonal cell line HCEC-B4G12 were obtained from Dr. Katrin Engelmann's lab (DSMZ, Braunschweig, Germany) [24]. They were cultured on culture flasks coated with 10 μg/ml laminin (Gibco) and 10 mg/ml chondroitin sulfate (Sigma). The medium consisted of Human Endothelial-SFM (Gibco) supplemented with 10 ng/ml basic fibroblast growth factor (bFGF, Gibco). The cells were cultured in 37° C., 5% CO₂ incubator until 90% confluence, after which they were passaged for experiments. The seeding density was kept at 6000 cells/cm².

After sterilization, PDMS substrates were coated with 10 μg/ml bovine fibronectin (Biological Industries) and 35 μg/ml bovine collagen I (Gibco) mixture, 0.2 ml/cm² of FNC Coating Mix® (US Biological), or 10 μg/ml laminin and 10 mg/ml chondroitin sulfate mixture for 2 hours at 37° C. They were then washed once with sterile filtered phosphate buffered saline (PBS, 1st BASE) and immediately seeded with cells.

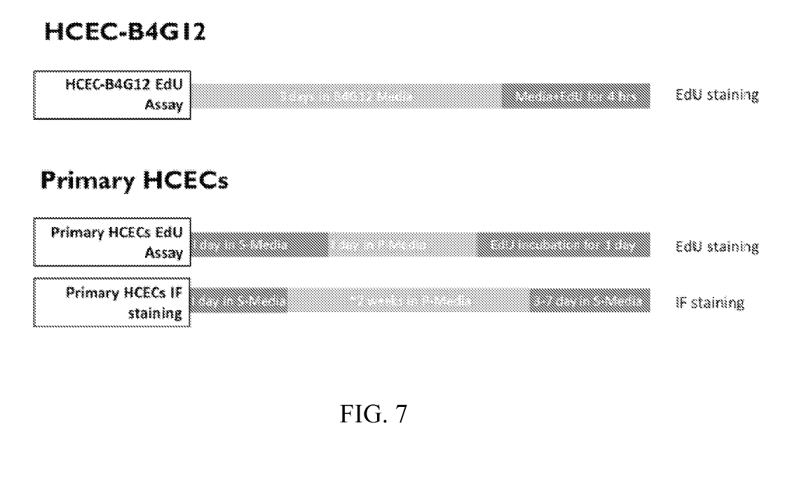

### Cell Culture Conditions for HCEC-B4G12 is Illustrated in FIG. 7

### Primary Human Corneal Endothelial Cells

Cell culture conditions for human corneal epithelial cells including primary cells are disclosed in WO2014014419 which is incorporated by reference in its entirety. Cell culture conditions for primary corneal epithelial cells include the following cell culture medium:

Stabilization media S-Media: Human Endothelial SFM, 5% FBS, 1% antibiotic-antimycotic

Proliferation P-Media: Ham's F12/M199, 5% FBS, 20 μg/ml ascorbic acid, 1× ITS (Insulin,Transferrin, Selenium), 1× antibiotic/antimycotic,10 ng/ml bFGF. The cell culture conditions are illustrated in FIG. 7.

### Immunofluorescence Staining Analysis of Morphology, BrdU Proliferation Assay, ZO1 and Na+/K+ ATPase

For the initial geometry screening, the cells were stained using standard immunostaining protocols after one week of culture. Briefly, cells were fixed with 4% paraformaldehyde (Sigma Aldrich) and permeabilized with 0.05% Triton X and 50mM glycine (Sigma). They were then blocked with 1% BSA and 10% goat serum for one hour at room temperature followed by incubation with the primary antibodies overnight at 4° C. Cells were immunofluorescently labeled for focal adhesion protein vinculin, while the nucleus was counterstained with 4′,6-diamidino-2-phenylindole (DAPI) and phalloidin for the cells filamentous actin (F-actin). Images of three patterned and unpatterned PDMS each were analyzed using an epi-fluorescent microscope (Leica DM IRB) and analyzed using Image J software (National Institute of Health). Cells stained for F-actin using phalloidin were used to investigate cell density, area, and circularity while DAPI stained cells were used to evaluate the cell numbers on the various patterns. Cell density data was expressed in average cell number/mm2 while circularity is defined as 4π (area/perimeter²). A circularity value of 1.0 indicates a perfect circle. As the value approaches 0.0, it indicates an increasingly elongated polygon. The average cell area and cell circularity for each pattern were analyzed using Image J software (National Institute of Health).

In order to study the response of the BCECs and the BCE monolayer to substrate topography, samples were fixed and stained after three and six days of culture. For cell proliferation and morphology studies, a lower seeding density was used to reduce the interference of cell-cell contact inhibition on the proliferation rate and to allow visualization of morphology of single cells for a more accurate measurement, respectively. The BCECs seeded at 2500 cells/cm2 were immunofluorescently labeled for 5-bromo-2-deoxyuridine (BrdU) and F-actin respectively. For BrdU analysis, 0.5 μg/ml of BrdU was added into samples 4 hours before undergoing 4% paraformaldehyde fixation. After fixation, BrdU samples were treated with 4N hydrochloric acid followed by blocking and subsequent procedures according to the standard immunofluorescent protocol. BrdU incorporation percentage was calculated as the percentage of BrdU-incorporating nuclei with respect to the total number of nuclei. It reflects the population of dividing cells and is proportional to the proliferation rate of the cells.

The monolayers were also immunofluorescently stained for tight junction protein Zona Occludens (ZO1). ZO1 stained images were used for cell area and coefficient of variation (CV) analysis of the BCEC monolayer. CV is defined as the ratio of standard deviation to the mean. For cell area and circularity analysis, a minimum of 50 cells per sample were analyzed. For BrdU analysis, a minimum of 700 cells per sample were analyzed, while for CV and area on monolayer, a minimum of 150 cells per sample were analyzed.

Samples immunostained for Na+/K+-ATPase pump followed the standard immunostaining protocol previously mentioned but were not subjected to the permeabilization step. BCE monolayers immunofluorescently stained for Na+/K+-ATPase were analyzed using confocal microscopy (Olympus, FV10-ASW). Images of random regions of each sample were photographed and analyzed using Image J software.

Primary antibodies used included: Mouse IgG Anti-Vinculin antibody (Sigma Aldrich) diluted 1:400, Mouse IgG Anti-ZO1 antibody (ZYMED laboratories) diluted 1:30, mouse IgG anti-Na+/K+-ATPase antibody (Developmental Studies Hybridoma Bank) diluted 1:360, mouse IgG anti-BrdU antibody (Developmental Studies Hybridoma Bank) diluted at 1:50. Anti-Mouse IgG Alexa Fluor 546 (Invitrogen) or anti-mouse IgG Alexa Fluor 488 (Invitrogen) diluted 1:750 was used as secondary antibodies while Oregon Green® 488 or Alexa Fluor® 546 Phalloidin (Invitrogen) was used for filamentous actin staining. All samples were counterstained with DAPI and mounted using ProLong Gold Antifade mounting medium (Invitrogen).

### Scanning Electron Microscopy (SEM) Imaging

On the 3rd day and 6th day of culture for the 60,000 cells/cm2 and the 40,000 cells/cm2 seeding densities respectively, the BCE monolayers were fixed with 2% glutaraldehyde in 0.1 M sodium cacodylate (Sigma) and 3 mM calcium chloride (Sigma) at room temperature for one hour. After rinsing in 0.1M sodium cacodylate and 3mM calcium chloride thrice for 5 minutes each, the samples were postfixed in 2% osmium tetraoxide for 1 hour and dehydrated in ethanol solution gradient. A final dehydration in 100% ethanol solution was carried out thrice for 5 minutes each before the samples were subjected to Critical Point Drying (Balzers Critical Point Dryer 030). The samples were then gold coated by ion sputtering (JEOL JFC 1600 Fine Gold Coater, 90 sec, 10 mA) before examination under a scanning electron microscope (SEM, Quanta FEG 200, HV mode) at an accelerating voltage of 10 kV.

### mRNA Expression Profiles

Total RNA was isolated from BCE monolayer at day 6 using TRIzol Reagent (Invitrogen). The RNA isolated from BCEs cultured on unpatterned PDMS served as controls. Synthesis and amplification of DNA were performed with the Qiagen one-step RT-PCR kit. Five pairs of primers were tested and their sequences are listed in supplementary table 2. Bovine Glyceraldehyde 3-phosphate dehydrogenase (GAPDH) was used as the endogenous control. Thirty cycles of PCR were carried out with an annealing temperature of 55 C and incubation for 30s. PCR reactions were resolved on 1.2% agarose gel in TAE buffer.

The densitometry analysis of the resolved RT-PCR bands was measured using ImageJ. The data were normalized to the GAPDH and then the normalized unpatterned control.

### Fluorescence Staining of Surface ECM

PDMS were washed with absolute ethanol and plasma treated before coating with either of the ECM. After 2-hour incubation, the samples were washed once with PBS and incubated with 1 μg/ml of Alexa Fluor® 546 Carboxylic Acid (Invitrogen) overnight. The samples were then washed with PBS and imaged.

### Immunofluorescence Staining of Na⁺/K⁺-ATPase and Tight Junction Protein 1

Three days after formation of confluent monolayer, the cell samples were fixed with 4% paraformaldehyde. They were permeabilized with 50 mM glycine and 0.05% Triton X-100. The permeabilization step was excluded for Na⁺/K⁺-ATPase surface marker staining. The samples were then blocked in 10% goat serum in PBS. Tight junction protein zona occludens 1 (ZO-1) was labeled with 3 μg/ml of mouse ZO-1 monoclonal antibody (BD Transduction) while Na^{+/}K⁺-ATPase was labeled with 5 μg/ml of mouse monoclonal IgG1 anti Na⁺/K⁺-ATPase al antibody (Santa Cruz). The samples were then incubated with 2.67 82 g/ml of Alexa Fluor 546®-conjugated goat anti-mouse antibody (Invitrogen) and counterstained with DAPI.

### Gene Expression Analysis

Gene expression of Na⁺/K⁺-ATPase and ZO-1 was quantified with real-time polymerase chain reaction (qPCR). The cells were cultured as above and RNA was isolated with TRIzol® Reagent (Life Technologies). For qPCR, TaqMan assay was run on Applied Biosystems 7500 Fast Real-Time PCR Systems using TaqMan® Universal MMIX II with no UNG.

TaqMan® gene expression assay ID Hs00167556_m1 and Hs01551861_m1 were used to detect Na⁺/K⁺-ATPase and ZO-1, respectively. Human glyceraldehyde 3-phosphate dehydrogenase (GAPDH) with assay ID Hs02758991_g1 served as control. Expression levels across different samples were first normalized against their GAPDH control, after which they were normalized against the unpatterned control.

### Protein Expression Analysis

HCEC-B4G12 cells were cultured as above and the protein was extracted three days after confluent monolayer formation with RIPA buffer (Pierce) containing protease inhibitor cocktail (Sigma) diluted 1:100, 1 μg/ml phenylmethylsulfonyl fluoride (Sigma) dissolved in methanol, and 1 μg/ml sodium orthovanadate (Sigma) dissolved in DI water. The samples were centrifuged and the supernatant containing the protein was extracted. The protein was then concentrated with centrifugal filter units (10K, Amicon) and quantified with Micro BCA Protein Assay (Pierce). The samples were then run on precast polyacrylamide gel (4-15%, Bio-Rad) and transferred to PVDF membranes (Bio-Rad) before blocking in filtered 15% bovine serum albumin (BSA) and incubation with either 0.91 μg/ml of anti Na^{+/}K⁺-ATPase α1 (Merck Millipore) or 2 μg/ml of mouse ZO-1 monoclonal antibody (Novex). They were then incubated with goat anti-mouse HRP (Santa Cruz). The proteins of interest were detected with ECL Reagents (GE Healthcare) on X-ray film (Fujifilm). Densitometry analysis was performed on the bands by normalizing each sample against GAPDH control, after which it was normalized against the unpatterned control.

### Morphometric Measurement

As ZO-1 expression is localized at the cell border and thus outlines the cell shape, ZO-1 immunostained images were used to calculate the individual cell circularity and area in ImageJ. Using the cell area values of 100-200 cells within a sample, coefficient of variation (CV) of cell area was obtained by dividing standard deviation of cell area with the mean cell area of that sample. The value of CV of cell area provides a quantitative index of polymegathism.

### Measurement of Na+/K+-ATPase Activity

The Na+/K+-ATPase activity was measured using the colorimetric Baginski Method [30]. Briefly, cell homogenate was obtained from the BCE monolayer in the reaction buffer (130 mM NaCl, 20 mM KCl, 4 mM MgCl2, 3 mM ATP, 30 mM histidine, in deionized water at pH 7.4), with or without the addition of 1 mM of ouabain. The ouabain was added to inhibit the pump activity, which served as the control samples. Enzymatic hydrolysis of ATP was stopped with the addition of 0.5 ml reagent A (2.857% w/v ascorbic acid and 0.4762% w/v ammonium heptamolybdate solution in 0.5 N HCl) to 1 ml of test solution and the sample was cooled to 4° C. for at least 6 minutes. The sample was then brought to 37° C. and 1.5 ml of reagent B (20 g sodium-m-arsenite and 20 g trisodium citrate-2-hydrate in 980 ml water and 20 ml acetic acid) was added. After 10 min incubation at 37° C., the reaction was complete and the sample was then brought to room temperature. The absorbance at 850 nm was read against a water blank sample and the amount of inorganic phosphate produced was estimated from a calibration curve of an inorganic phosphate standard in the range 10 to 100 nmol phosphate per sample. Specific Na+/K+ ATPase pump activity was calculated using the difference between the amounts of inorganic phosphate liberated in presence or absence of ouabain. The protein concentration of the reaction mixtures was determined by the Bicinchoninic acid assay (BCA, Pierce) with a minimum of triplicates for all samples.

### Data Analysis

All data are presented as mean±SD unless specified. Student's t-test and one way ANOVA and Bonferroni post-test correction were used to evaluate the statistical significance where indicated.

### EXAMPLE 1

### Surface Characterization of Fabricated Substrates

Microscale patterns fabricated using soft lithography retained their integrity as observed using SEM (FIG. 1). The fabrication technique was robust for even the nano-scale patterns

(FIG. 1L-M). The structure size was consistent, and the actual dimensions, which were measured by SEM or cross-sectional SEM, were within 15% of the intended dimensions of the mold design (Supplementary Table 1). Using SEM, it was observed that the coating of laminin did not adversely affect the integrity of the fabricated patterns. The laminin coated was relatively thin and smaller in size as compared to the topographical substrates (Fig. S1). Laminins are a family of glycoproteins that are an integral part of the structural scaffolding of basement membranes, and have specific peptide sequences to which focal adhesions of endothelial cells can bind to via ECM binding cell surface receptors like integrins. The common integrin binding sites on laminin-1 includes α1β1 and α2β1 binding sites (reviewed in [31]). In our study, the substrates were coated with laminin-1 to mimic the biochemical microenvironment of the basement membrane, and to enhance the cell-substrate interaction.

### EXAMPLE 2

### Screening of BCEC Response on Various Geometrical Micro-Scale Patterns

The aim of screening geometries was to investigate the different effects of substrate topography on cellular behaviour, in order to select topographies for detailed cell behavior characterization.

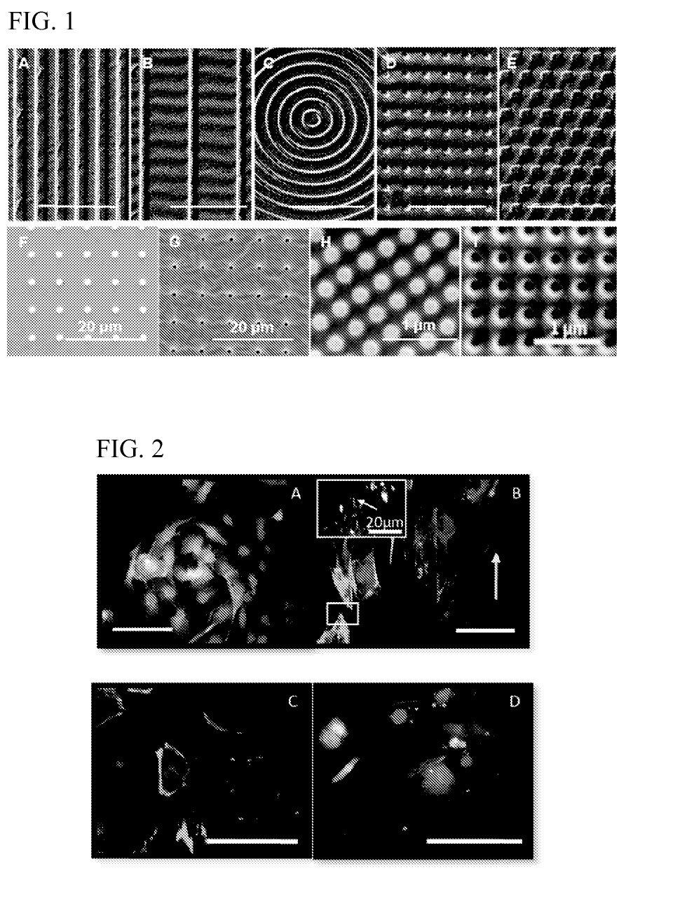

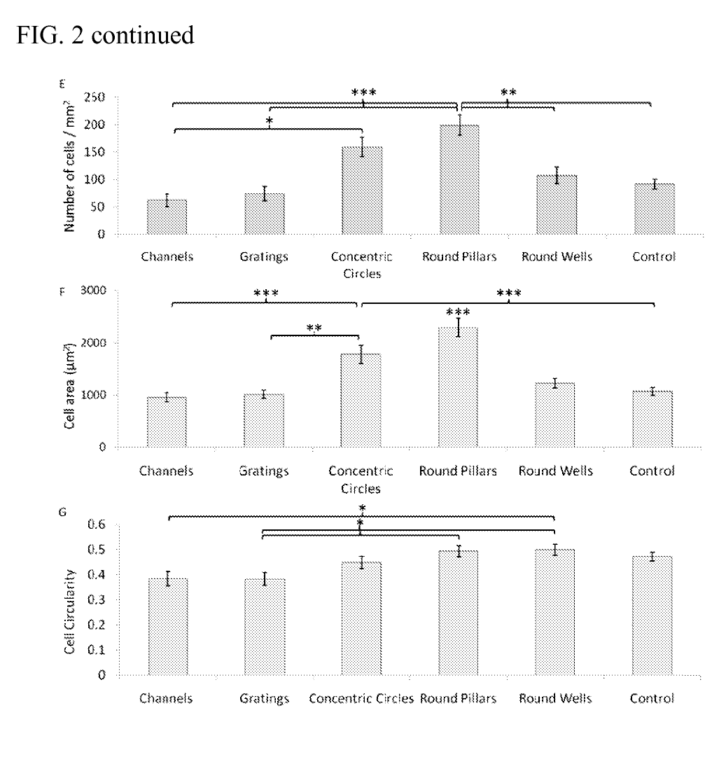

BCEs immunofluorescently stained for F-actin (green), DAPI (blue) and vinculin (red) are shown in FIG. 2A-D. Vinculin is a protein that is found in focal adhesions and is involved with the linkage of integrin adhesion molecules to the actin cytoskeleton. The role of vinculin in transducing topographical signals however remains unclear. Differences in both cell alignment and organization could be observed from the images of BCECs seeded on different patterns including concentric circles, gratings, pillars and unpatterned control. BCECs seeded on concentric circles were found to respond to the underlying circular topography through their alignment and organization. The cells aligned preferentially along the circumference of the concentric circles and proliferated in an organized pattern circulating the ridges, indicating that the underlying topography significantly affected the BCEC cell proliferation and development patterns. Similarly for the gratings, the cells aligned preferentially along the gratings (grating axis in yellow) and also exhibited some degree of elongation (FIG. 2B and Supplementary FIG. 2). This observation is similar to previous findings, where corneal epithelial cells cultured on micro and nanoscale grooves exhibited a preferential alignment and elongation in the direction of the grooves [19, 21]. It is important to note that these studies used human corneal epithelial cells on grating widths that ranged from 200 nm to 2 μm in width with two different depths, namely 150 nm and 600 nm. In contrast, when BCECs were seeded on pillars, they were found to spread out randomly and exhibited little or no alignment, indicating that these patterns substantially influence the morphology and proliferation patterns of BCECs.

Immunofluorescence images showed peripheral vinculin which could be observed at the ends of actin filaments in the cells at the leading edge (FIG. 2B insert). The almost parallel arrangement of vinculin and actin filaments co-localization (white arrow) on BCECs cultured on microscale gratings suggests that the underlying substrate topography determined the directionality of focal adhesion formation and in turn actin polymerization, although it is unclear which of these two events comes first. The BCECs adaptation to different morphologies on different topographies were indicative that BCECs do react to the underlying topography.

### EXAMPLE 3

### Cell Density, Area and Circularity of BCECs on Different Topographies

The average cell densities after one week of culture were estimated for the different substrate topographies used (FIG. 2E). The different topographies had varying cell densities, ranging from 62±34 cells/mm2on gratings to 199±65 cells/mm2 on pillars. The cell densities on the round pillars were found to be significantly higher than those on the gratings, channels, wells and the unpatterned substrates, suggesting that pillars induced higher proliferation rates for the BCECs as compared to the other patterns.

Cell densities on pillars were also found to be significantly higher than those on the wells. Interestingly, the two topographical structures were the inverse of one another bearing the same dimensions. This indicates that the increased proliferation rate is possibly induced by topographical effects since both patterns effectively have the same surface area of cell-substrate interaction. We believe elevated pillar structures allows for increased cell adherence compared to depressed well structures. The well structures may have also effected a reduction in initial cell adherence compared to the pillars. Observation at shorter time points, for example, hours after cell seeding, will be necessary to differentiate this outcome from the effects of topography on cell attachment. Nevertheless, enhancement in both cell adhesion and proliferation using the pillar structures would be beneficial for the cornea endothelium reconstruction.

A similar trend was observed in the average cell areas for the different topographies after one week of culture (FIG. 2F). Analogous to cell density observations, the cell area for the pillars was the highest, at an average of 2,296 μm2. The cell area for pillars were again significantly higher than many of the other patterns, including gratings, channels, wells and the unpatterned control. In addition, the cell areas found on pillars were also significantly higher than those found on wells. The similar trend with cell densities suggests that the underlying topography may influence cell proliferation through increased cell spreading. It is possible that topographies like pillars allow a higher degree of cell spreading, thus leading to an increased proliferation rate. This is coherent with previous observations where cell proliferation was dependent on the degree of cell spreading [32-34].

There were however, no significant differences found for the cell circularities among most of the patterns (FIG. 2G), except for channels and gratings, on which the BCECs were more elongated compared to other patterns such as pillars, wells or the unpatterned control.

### EXAMPLE 4

### Analysis of BCECs and the BCE Monolayer

Based on earlier studies [35] [36], cells are able to detect differences between micro and nano sized features. Our results from the geometry screening of micro patterns (FIG. 2E-G) also indicated preferential feature types. BCECs on pillars showed high cell density, cell area and circularity while the inverse well features showed otherwise. The topographies of 1 μm and 250 nm pillars, 1 μm and 250 nm wells were thus selected for further investigations. In addition to individual cell response, BCECs were cultured on these substrates and allowed to form a confluent monolayer before investigating them for cell densities, cell area regularity as well as the formation and localization of functional proteins/structures for the barrier and pump functions.

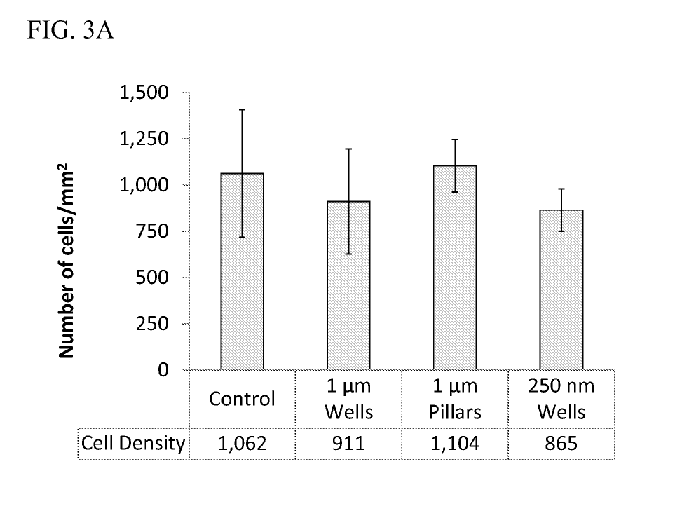

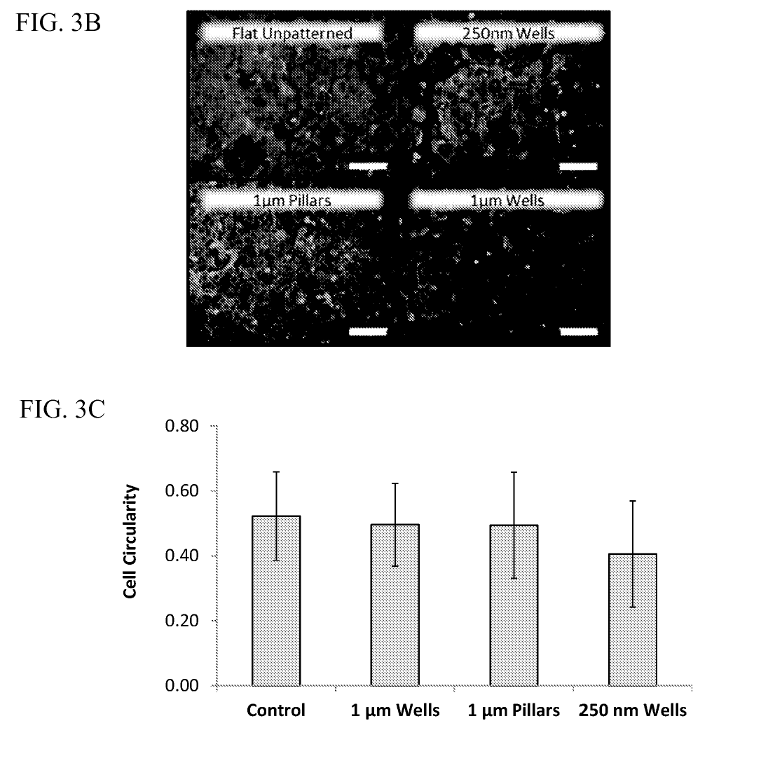

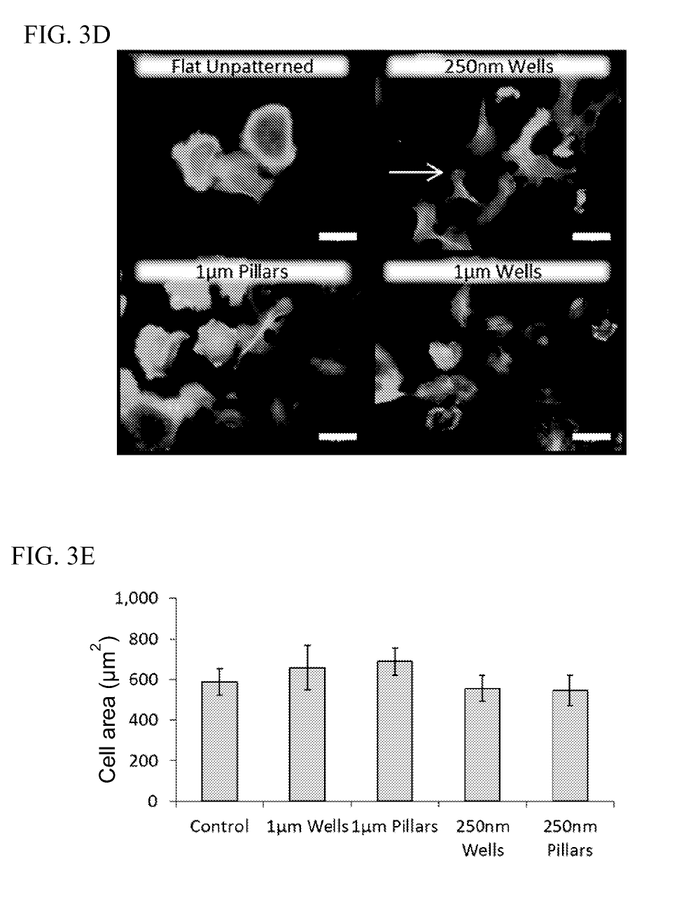

At a lower seeding density of 2500 cells/cm2, the BCECs were immunofluorescence stained and analyzed for cell density, area, circularity and BrdU incorporation. The average cell densities were quantified from four different substrates (FIG. 3A). The cell densities found on the flat substrate and micro pillars were found to be significantly higher than those found on the micro wells and nano wells, respectively. This is evident from images of the cells on the different substrates (FIG. 3B). The BCECs seeded on the micro pillars were already confluent and begun to form the polygonal formation found in native cornea endothelium as opposed to the other substrates. In contrast, the micro wells had the lowest cell density. These observations were similar to the results derived from the initial geometry screening study, where elevated topographies seemed to induce a higher proliferation rate as compared to depressed topographies like wells. We speculate that elevated features could aid the nucleation, formation or stabilization of focal adhesion, which would trigger the signals for cell spreading and cell proliferation.

When we measured the cell circularity of the BCECs (FIG. 3C), it was observed that BCECs on the 250 nm wells was found to be significantly lower than those found on the other three substrates. Observations from F-actin stained images indicated that the BCECs on the 250 nm wells were less circular and had lamellipodia and filapodia in random directions, assuming a more stellate morphology (FIG. 3D). This implied that the BCECs were more actively migrating on the nano-wells compared to the other substrates where they adopted a rounded morphology. A similar observation was found with human cornea epithelial cells cultured on micro and nanoscale wells in an earlier study [20]. Epithelial cells on non-pattered surfaces and microscale wells assumed a more evenly spread morphology while those on nanoscale wells had numerous protrusions extending from the main cell body. In addition, nanoscale wells had a lower cell density compared to unpatterned and microscale wells after a 24 hour incubation period, which was in accordance with our previous results for BCECs cell density. These results suggest that micro and nanoscale topographies have distinct effects on BCECs morphology and nano-sized pillars can induce higher BCECs proliferation rate.

In order to investigate the effect of topographical features and size, we first measured the average cell areas on the pillar and well substrates, both micron and nano-scaled features (FIG. 3E). We found that the cell area found on the micro pillars was higher than those on micro and nano-wells. Interestingly, the cell area was also higher than those on nano-sized pillars, although the difference was not significant. It was observed that increased cell area correlated with increase cell densities. Micron pillars supported cells with higher cell area and thus higher density. This observation was also evident in the initial geometry screening study. This suggests that a larger degree of cell spreading leads to an increase in cell proliferation for BCECs. It has been documented that cells need to spread in order to enter the S phase for DNA replication phase [37]. While the exact mechanism by which cell spreading influences cell proliferation is unknown, the link between cell proliferation and cell-substrate adhesion is evident in the ability of integrins to activate mitogenic pathways [37, 38]. Thus, it is possible that topographies like pillars allow for a higher degree of spreading compared to wells, resulting in the activation of mitogenic pathwaysand thus higher proliferation and cell densities on these patterns. Immunofluorescence images of cells on pillar like topography showed localization of focal adhesions on these elevated structures as compared to flat substrates (unpublished data), indicating increased levels of cell anchorage on these elevated patterned surfaces leading to improved cell proliferation compared to other geometry and unpatterned substrates.

The BCECs proliferation rates on different scaled size pillars and wells using BrdU incorporation were shown in FIG. 3F. The BrdU incorporation, indicative of BCECs proliferation were significantly higher on 1 μm wells, 1 μm pillars and 250 nm wells compared to the unpatterned control substrate. Interestingly, the proliferation rate was significantly higher on the 1 μm wells and lower on the 250 nm pillars. This is in opposition to the suggestion that the BCECs proliferate faster on the pillars and flat substrates as compared to the wells. This could be due to cell-cell contact inhibition, which reduces proliferative capabilities. We believe that since BCECs proliferate faster on micro pillars and on the unpatterned substrate, they reach confluence earlier on these substrates compared to well structures. The contact inhibition signals that the cells produce when they are confluent may reduce their proliferative capacity, thus explaining the similar levels of BrdU incorporation between the 1 μm well and 1 μm pillars.

### EXAMPLE 5

### Zona Occludens-1 (ZO1) Expression and Polygonal Cell Shape of BCE Monolayer

ZO1 is a tight junction-associated protein which plays a crucial role in maintaining the barrier function of the cornea endothelium. The shape of the native corneal endothelial cells of vertebrates is typically a mixture of hexagonal and pentagonal cells, in which the cell borders are irregular and interdigitating [39, 40]. The analysis of ZO1 expression and the geometries of a reconstructed endothelial monolayer are thus important considerations.

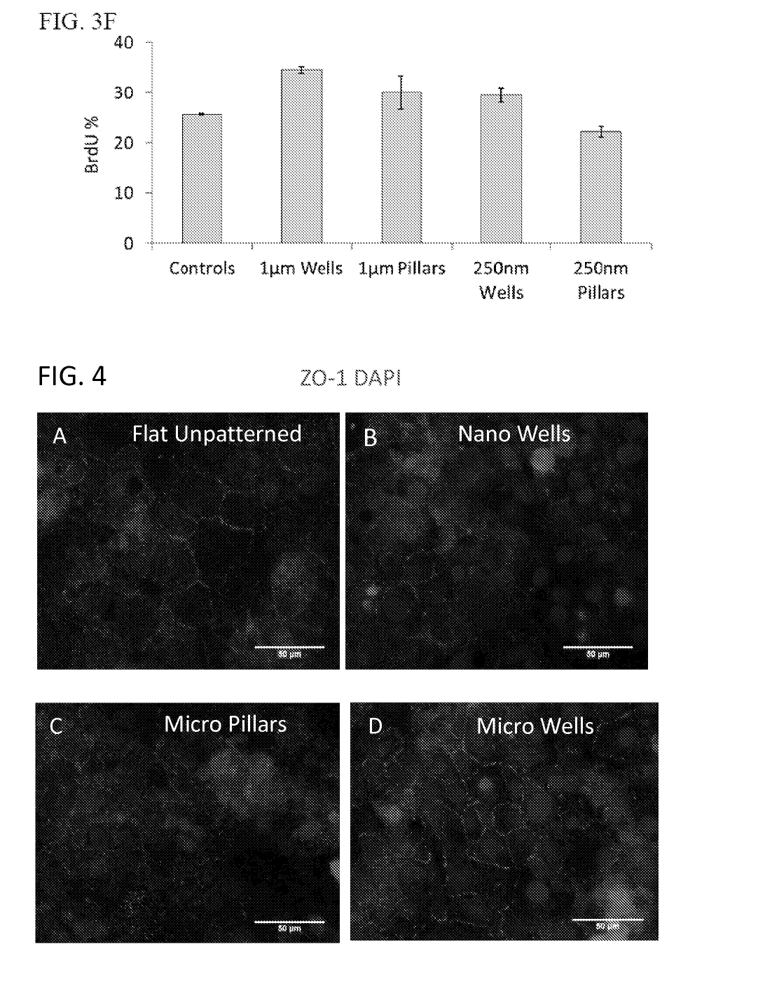

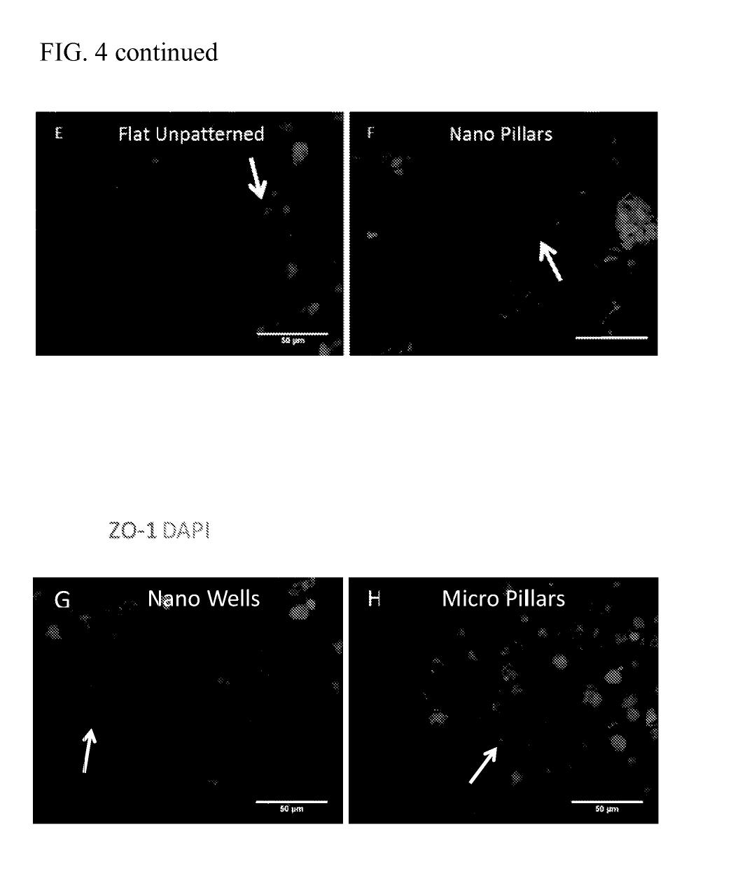

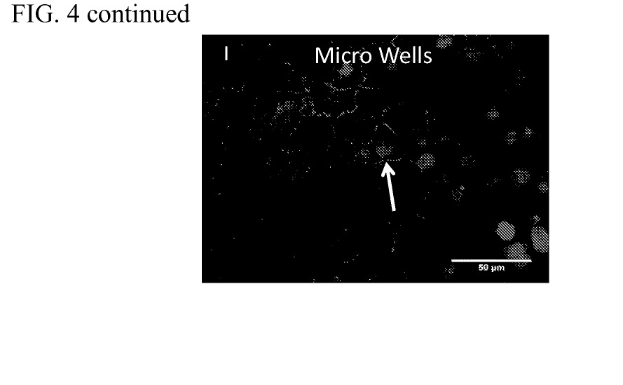

The images of immunofluorescently stained ZO1 BCECs were used to analyze cell shape and ZO1 expression. In order to investigate different periods of cell interaction with topography, BCECs were seeded on the substrates at two different densities 60,000 cells/cm2 and 40,000 cells/cm². These cells were then allowed to form a monolayer before fixing, which was on the 3rd and 6th day of culture respectively for the two different cell densities. The BCECs monolayer seeded at a higher density (3rd day, FIG. 4 A-D; the immunofluorescence images of ZO-1 and F-actin are shown in Supplementary FIG. 3) showed differences in both ZO1 expression and BCEC boundary establishment when cultured on different substrates. ZO1 proteins were observed to be expressed along the cell boundaries, indicating the formation of tight junctions in the BCEC monolayer with higher expression on micro wells as shown in FIG. 4D.

For the BCECs seeded at a lower density of 40,000 cells/cm2, the monolayer was fixed on the 6th day and similarly stained for ZO1 (FIG. 4 E-I, the immunofluorescence images of the ZO-1 and F-actin are shown in Supplementary FIG. 3). On all substrates, a portion of cells that formed the monolayer were polygonal in shape, while others were adopting a hexagonal shape, coupled with the distinct expression of ZO1 proteins on the boundaries of these cells.

The observations from the BCEC cell shape and ZO1 expression suggests that the topography-induced signal can remodel and maturate the BCECs with time. This is more evident on the micro and nano-scaled wells substrates, where cell boundaries and ZO1 expression were particularly distinct on some cells after 6 days of culture using lower seeding density (FIG. 4G and I), compared to the monolayer after 3 days of culture using higher seeding density (FIG. 4B and D). The use of substrate topographies for BCECs maturation will be an important consideration in future studies and applications. A more in depth study documenting the time response of BCECs to topographies will aid the design of tissue engineered corneal constructs.

The polygonal shape observed in the ZO1 stained samples is typical of both the native bovine and human cornea endothelium [39, 40]. The BCECs in the cultured monolayer exhibited a range of geometries, ranging from four-sided to seven-sided cells. The human corneal endothelium does not, however, have a constant organization and can vary from having a uniform appearance (with most cells being of similar size) to a non-uniform appearance (with some or many cells being much smaller or much larger than the rest). In one study of 20 healthy young adults, the observed endothelium was composed of a mixture of 4-, 5-, 6- and 7-sided cells, where the 6-sided cells was relatively highest at 61.3%. The rest of the cells were either 4-sided (1.4%), 5-sided (19.6%), 7-sided (16.4%) or 8-sided (1.4%) [41]. The observed range of number sided cells in the BCE monolayer thus might not be a sign of poor functionality.

### EXAMPLE 6

### Resemblance to Native Cornea Endothelium: Monolayer Cell Area Coefficient of Variation (CV) and Microvilli Formation

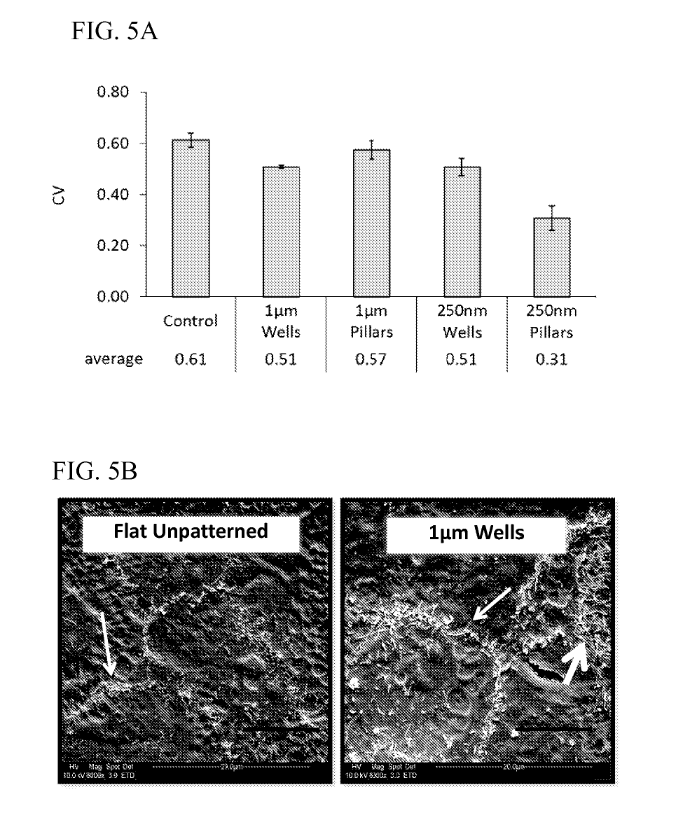

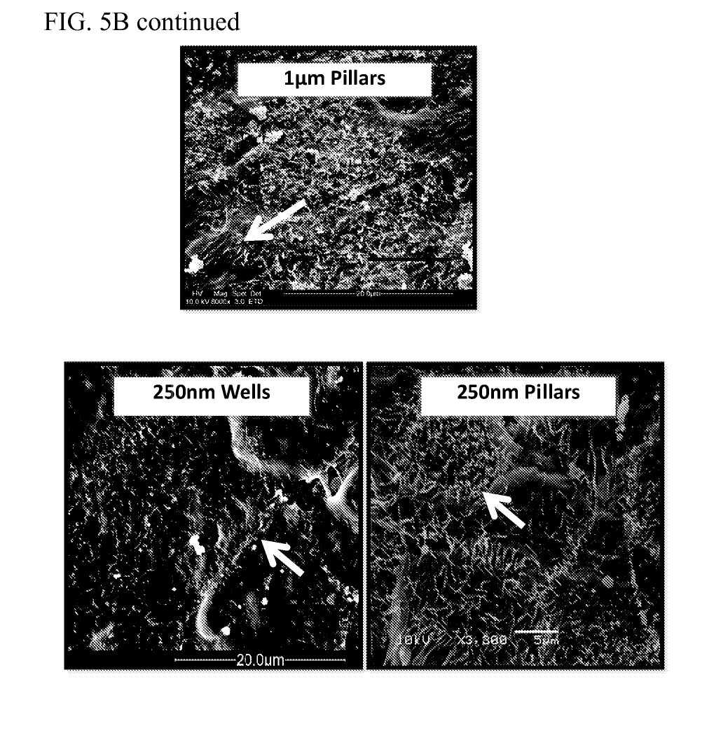

Cell boundaries of the 6th day BCE monolayers were outlined for cell area measurements using ImageJ. CV of mean cell area was calculated as percentage of standard deviation (SD) with respect to mean cell area for each sample (FIG. 5A). The CVs of BCECs ranged from 0.31 on the 250 nm pillars, 0.51 on the 250 nm wells and 1 μm wells, to 0.61 on the unpatterned control. The CV of BCECs on 250 nm pillars was significantly lower than all other substrates. A healthy and functional native corneal endothelium consisting of uniform cells is expected to have a CV of 0.25. A study with 15 healthy adults with an average age of 21.7 years indicated an average CV of 0.315 [40]. CV of mean cell area is a clinically valuable marker used to detect polymegathism, a term used to describe an increase in variability of the cell area and is an indicator of corneal endothelium wellbeing. It is also currently the most sensitive index for corneal endothelial dysfunction [2]. The CV measured on the nano-pillars was within the range of a healthy cornea endothelium. While the CVs on the other substrates were significantly higher those found in the native corneal endothelium, BCECs on the wells and pillars exhibited a reduced CV value compared to unpatterned control. The results suggest that the underlying substrate, particularly the nano-pillars, could induce a more regular distribution of cell area in the BCE monolayer. Scanning electron microscopy showed the presence of surface microvilli (FIG. 5B) on the BCE monolayer. The presence of microvilli and cilia in the native human cornea endothelium has also been documented [6, 39, 41, 42]. The formation of cilia is important for increasing the surface area of the cell to facilitate the pump function of endothelium [42]. The microvilli observed in this study appeared to be denser in the monolayers seeded on the micro- and nano-pillars as compared to the other three substrates. A high microvilli density could also be observed on the cell boundaries on all substrates. The higher density of microvilli found on the pillars may indicate that pillars or elevated topographies induce these cells to exhibit more in vivo like properties. It has been demonstrated that native cornea endothelial cells usually possess more microvilli than do cultured cornea endothelial cells [40]. Thus, the higher density of microvilli present on the BCE monolayer formed on these pillars will be a relevant consideration. Although the cornea endothelium microvilli function have yet to be determined, it has been postulated that the increased surface area provided by the microvilli may aid in ion transport associated with endothelial stromal dehydrating mechanism. In this dehydrating mechanism, both bicarbonate and sodium ions are transported from the stroma to the aqueous humor, creating an osmotic gradient that balances the swelling tendency of the corneal stroma [43]. Thus, enhanced microvilli observed onpillar substrates may indicate improved functionality in BCECs. On the other hand, BCE monolayer cultured on nano wells showed the lowest density of microvilli, thus suggesting that the topographic cues presented by the nanoscale wells may play a role in reducing microvilli density.

### EXAMPLE 7

### Expression and Function of Na+/K+-ATPase Pump

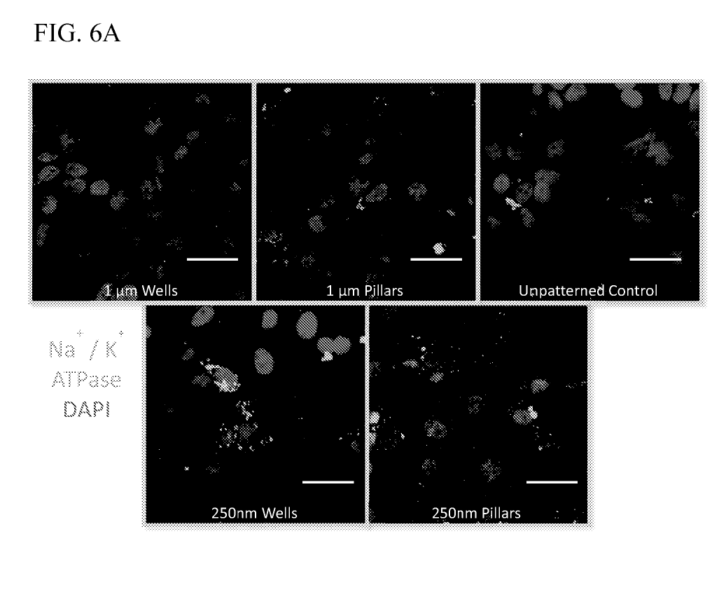

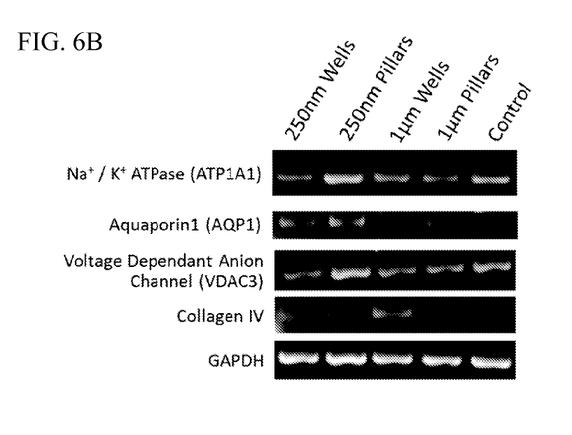

One important function of the corneal endothelium is to facilitate hydration of the stroma through ionic pump activity. This pump function prevents stroma swelling by removing excess stromal fluid to maintain optical transparency. It is generally agreed that Na+/K+-ATPase activity is coupled to the fluid pump function of corneal endothelium [2, 44]. Immunofluorescence staining was used to verify the presence of Na+/K+-ATPase pump activity (FIG. 6A and Supplementary FIG. 4). Positive staining of Na+/K+-ATPase was observed on all topographies; however, the staining intensity was higher in the micro and nano pillars and the nano-wells, compared to the 1 μm wells and the unpatterned controls.

To verify our immunostaining results, semi-quantitative RT-PCR analysis showed higher expression of Na+/K+-ATPase. In addition, other genes of membrane transport proteins such as Aquaporin 1 (AQP1) and Voltage Dependant Anion Channels (VDAC3) were observed in the BCEC monolayer cultured on the nano-pillars (FIG. 6B and the densitometric analysis of the semi-quantiative RT-PCR in Supplementary FIG. 5). AQP1 is essential for osmotically driven water transport [45] and VDACs are essential for anion transport [46, 47]. The upregulation of the membrane transport protein gene expression suggested that nano-pillars could enhance the pump functionality of the BCEC monolayer.

In addition to the membrane transport protein related gene, the expression of collagen IV was also examined. Collagen type IV is one of the major types of collagen found in the Descemet's membrane secreted by corneal endothelial cells [48, 49]. The expression of collagen IV has been found to be altered in most immortalized corneal endothelial cell lines [50]. The RT-PCR result showed a general upregulation of the collagen IV gene expression in the BCEC monolayer cultured on the micro- and nano-well and pillars, compared to the unpatterned control. The transcriptional expression of collagen IV suggested that substrate topography maintained the extracellular matrix secretion properties of the cornea endothelial cells in vitro.

Na+/K+-ATPase activity was quantitatively measured using the Baginski method, which was based on evaluating the difference between the amounts of inorganic phosphate liberated in the presence or absence of the pump inhibitor ouabain. As pump activity assay required a large amount of samples in order to obtain reliable result, the test was limited to the pattern on which BCE exhibited the most optimized phenotypic outcome, hence the 250 nm pillars. The 250 nm pillars pattern was chosen because it induced enhanced phenotypes of BCE in terms of higher cell density, more regular cell area, lower CV of cell area, as well as higher microvilli density in addition to higher expression of the pump as indicated by immunofluorescence-stained images and RT-PCR. The phosphate liberated from the BCEC monolayer cultured on 250 nm pillars was 0.205±0.024 μM/mg/10 min compared to 0.104±0.051 μM/mg/10 min on the unpatterned control (p<0.05, student's t-test). The measured Na+/K+-ATPase activity was comparable to the hydrolyzed ATP value measured in mice [51], human [8] and bovine [52] corneal endothelial layer. Our results emphasized that nanotopography in the form of pillars could enhance the expression and function of Na+/K+ ATPase pump. It will be beneficial to enhance the pump function of the BCEC monolayer using nanotopography as opposed to stimulatory chemical agents (e.g Dexamethasone) that has harmful side effects. Our proof-of-concept study using BCE cell line as cell model again indicated the impact that topography has on corneal endothelial cell behavior. The findings in the current study would provide knowledge for future in vitro studies with other primary corneal endothelial cells or in vivo studies.

### EXAMPLE 8

Primary HCECs isolated from patients' samples were cultured and tested on either FNC coated or laminin-chondroitin sulphate coated (LC) TCPS with or without patterning by using dual-media approach. The experimental protocol is shown in FIG. 7 for proliferation assay and HCEC marker staining where S-Media represents stabilizing media and P-Media represents proliferation media.

### EXAMPLE 9

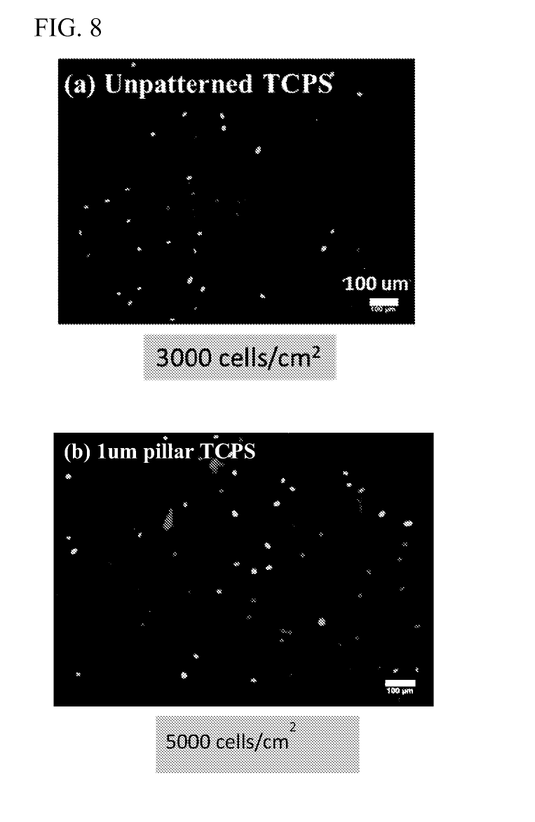

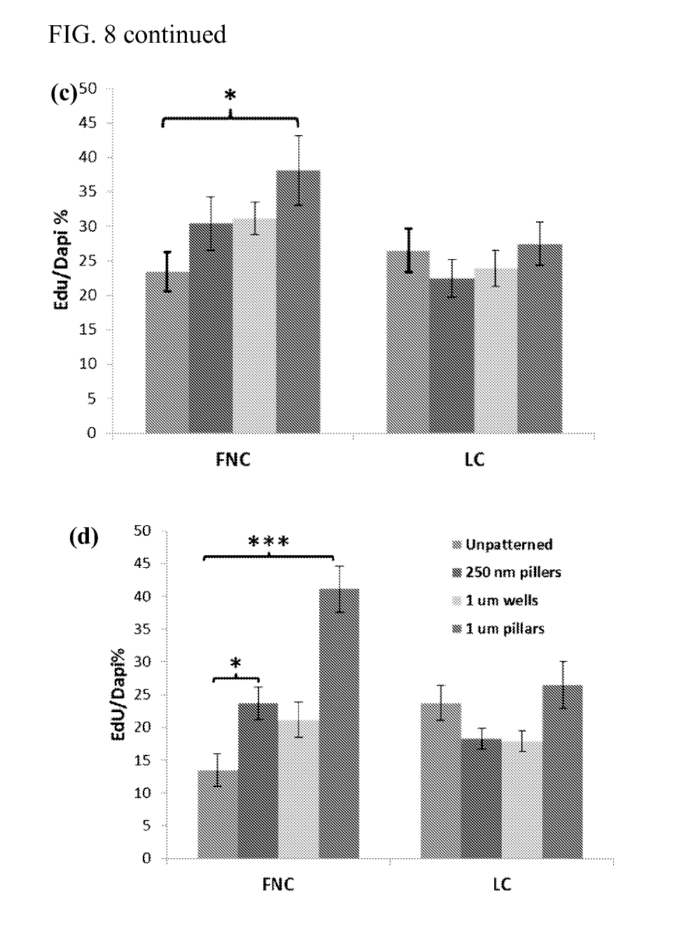

Primary HCECs proliferation was measured by incubating cells with EdU dye. EdU dye is incorporated in the proliferating cell's DNA and thus, it can be used to evaluate cell proliferation. FIG. 8 shows the effect of patterned TCPS topography on the proliferation rate of HCECs at two different seeding densities. Proliferation rate of HCECs was not significantly different on various substrates on LC-coated samples. However, on FNC-coated samples, proliferation rate of HCECs was found to be significantly higher on 1 μm pillars as compared to unpatterned TCPS control at both seeding densities. However, at 5000 cells/cm² seeding density, the difference of the proliferation rate between 1 μm pillars and unpatterned control was much higher as compared to 3000 cells/cm² seeding density. 250 nm pillars did not show significantly different proliferation rate as compared to control at 3000 seeding density but it was significantly higher at higher seeding density.

These results of topography interaction with HCECs to regulate the proliferation rate in combination with the ECM coating are promising and provide evidence that proliferation can be upregulated with the use of surface topographies without adding extra components in the culture media which could affect HCEC cell phenotype.

### EXAMPLE 10

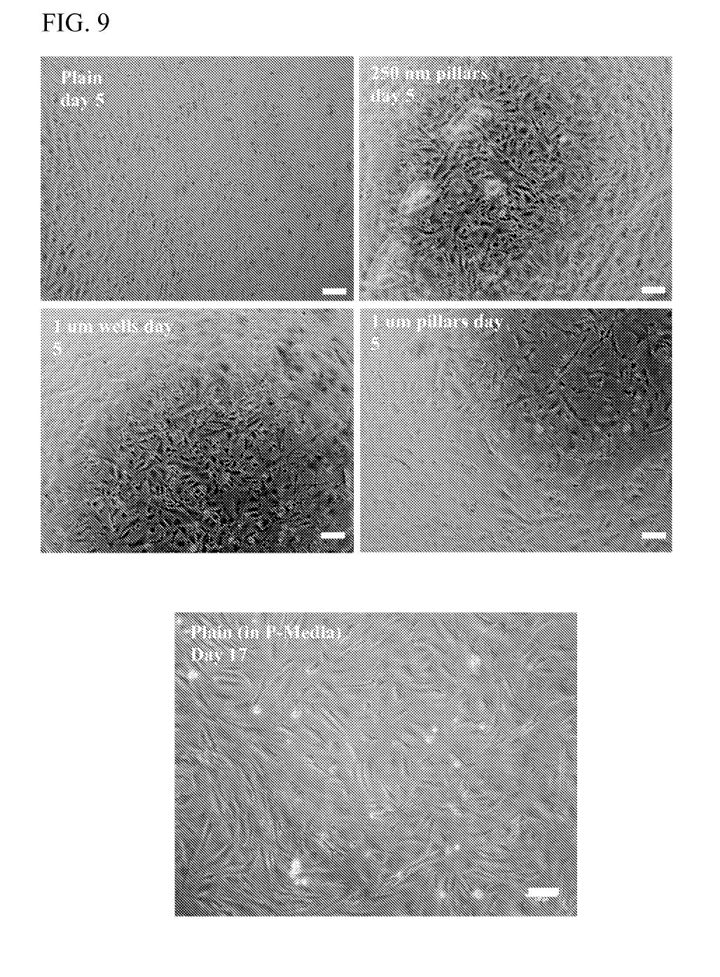

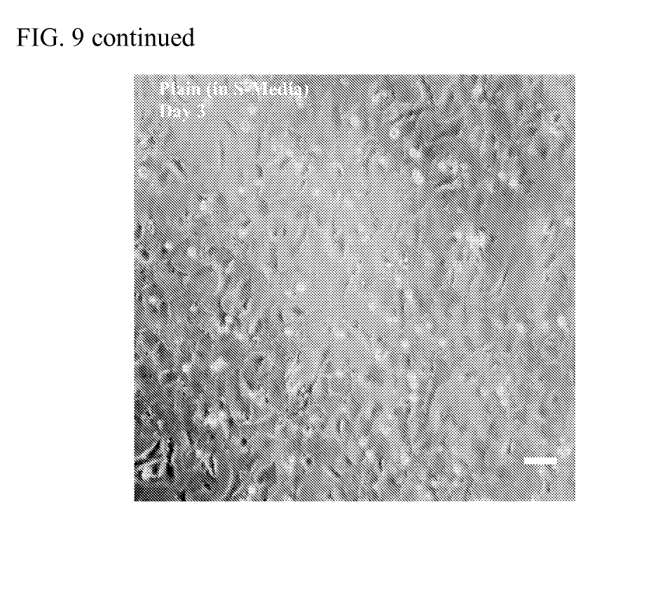

FIG. 9 shows representative phase contrast images of HCEC cell growth on TCPS topographies at day 5 and it also shows the difference in cell morphology in proliferation media (P-Media) at confluency and in stabilization media (S-Media) at day 3. HCECs in S-Media are more rounded and polygonal in shape while in P-Media, the cells are elongated. Enhanced proliferation rate of cells in P-Media could be associated to the cell morphology.

### EXAMPLE 11

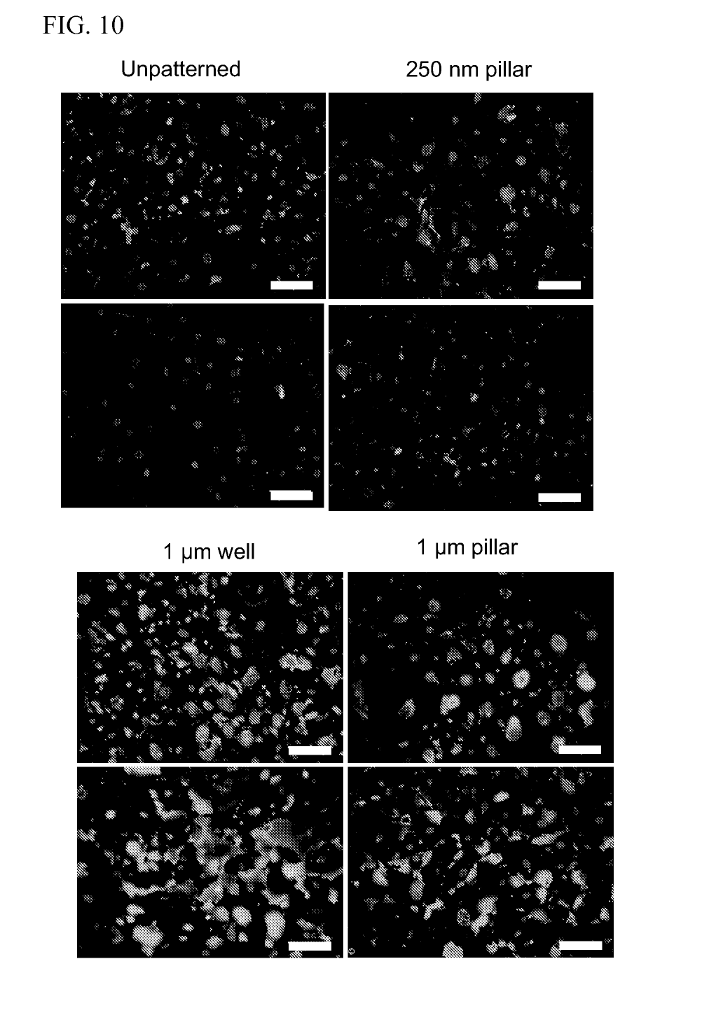

FIG. 10 shows the expression of Na⁺/K⁺-ATPase as expressed by primary HCECs after reaching confluency. All images represent primary HCECs on patterned TCPS with FNC and laminin & chondroitin sulphate (LC) protein coatings. HCECs maintain their phenotype and showed generally good expression of Na⁺/K⁺-ATPase on all surfaces. The results suggest that the current protocol for the expansion of HCECs did not alter the expression of Na⁺/K⁺-ATPase of HCECs.

### EXAMPLE 12

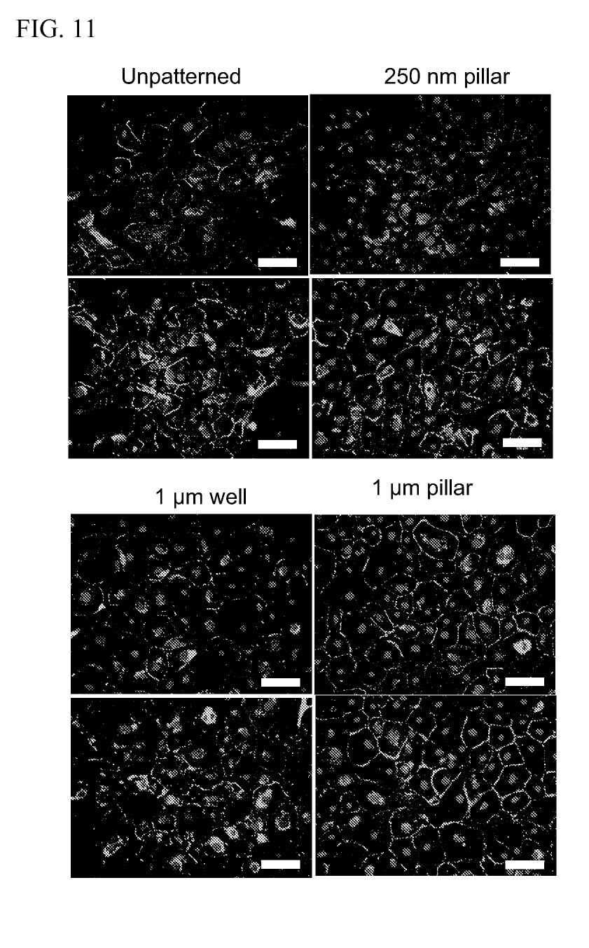

FIG. 11 shows the expression of tight junction protein ZO-1 as expressed by primary HCECs after reaching confluency. All images represent primary HCECs on patterned TCPS with FNC and laminin & chondroitin sulphate (LC) protein coatings. HCECs maintain their phenotype and showed generally good expression of ZO-1 functional marker on 1 um pillars with either coating (FIG. 11). The results suggested that the 1 μm pillar is favourable for proliferation and maintaining the cell function for primary HCECs.

### EXAMPLE 13

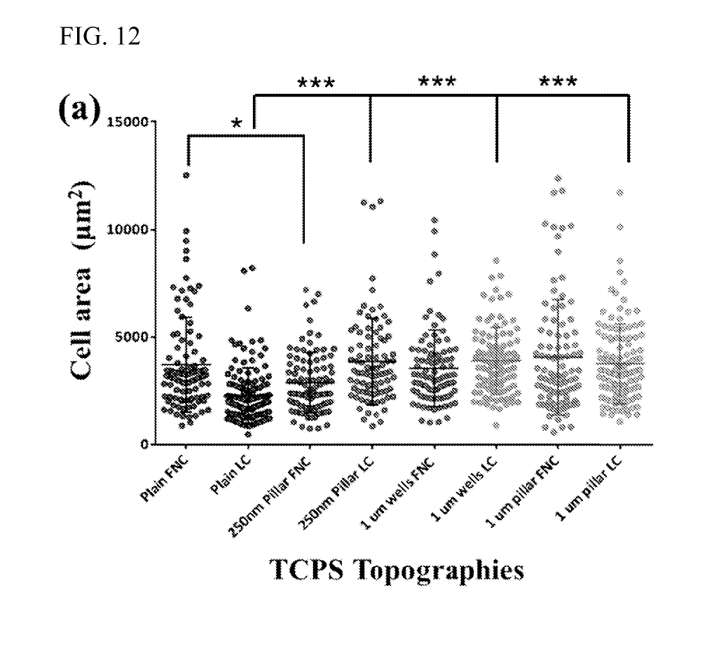

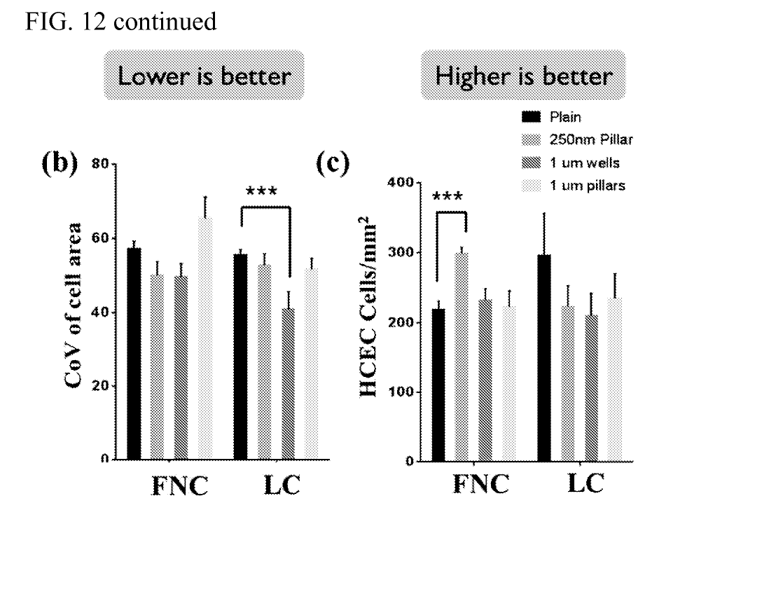

Primary HCEC cell shape was analysed by using ImageJ software which included cell area, coefficient of variation (CoV) of cell area and number of cells/mm2. FIG. 12 shows the effect of various protein-coated patterned TCPS topographies on the distribution of cell areas in a dot plot where each dot represents a cell. Cells on LC coated TCPS topographies expressed wide distribution of cell areas as compared to unpatterned TCPS where cells had significantly lower cell areas with much tighter distribution. On FNC coated surfaces, 250 nm pillars had significantly lower cell areas with much tighter distribution compared to unpatterned controls. HCECs cell area has important consequences because lower cell areas are desired due to the fact that primary HCECs in CE monolayer in-vivo has cell areas in 400-600 μm2 range [98]. In the current experiment, although the HCEC cell areas are an order of magnitude higher because of low seeding densities as compared to in-vivo monolayer cell density, still it provides information that topography is able to modulate HCECs area.

FIG. 12(b) shows the effect of protein-coated TCPS topographies on Coefficient of Variation (CoV) of cell areas. On FNC coated samples, 250 nm pillars had lower CoV as compared to control. The lowest CoV was found on LC coated 1 μm wells. Here it is worth mentioning that the CoV of HCECs in-vivo is around 0.26 which shows even tighter distribution of cell areas in-vivo.

Finally the numbers of cells per mm² area are calculated and compared with in-vivo CE monolayer cell densities. As FIG. 12(c) shows, FNC coated 250 nm pillars had significantly higher cell densities of confluent monolayer as compared to unpatterned controls. While on LC coated TCPS, control samples were found to have higher cell densities. Higher cell densities are required as there is a threshold value of 250-1000 cells/mm² for the HCEC monolayer cell sheet to function properly.

### EXAMPLE 14

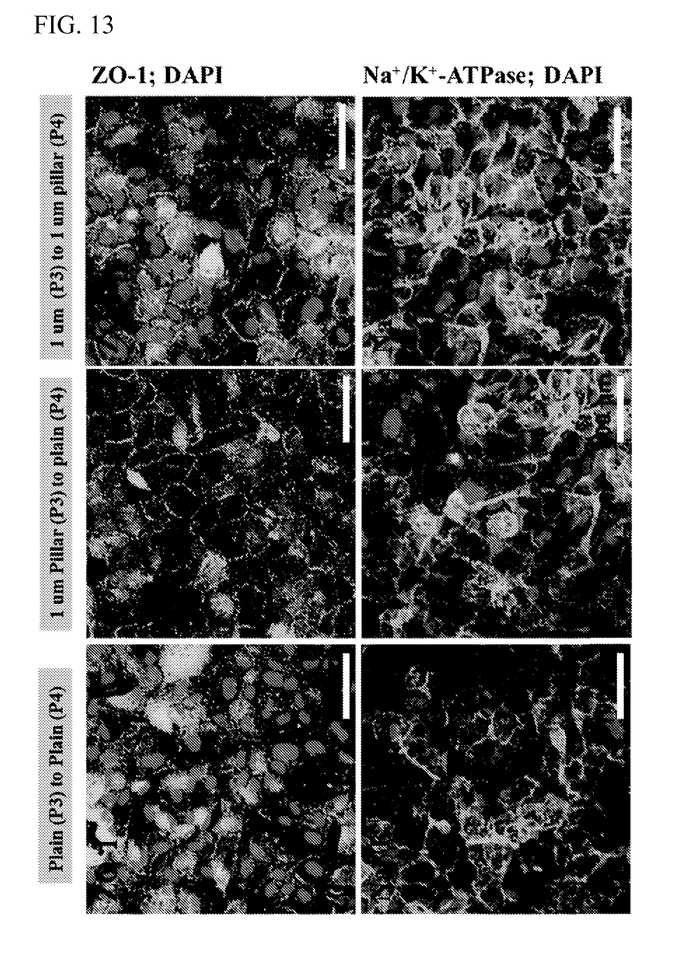

The capacity of the primary HCECs cells was assessed for forming an intact monolayer and for maintaining their phenotype after they have been grown on patterned TCSP at P3. For the preliminary studies, 1 μm topography was selected to study as the HCECs demonstrated highest proliferation rate on this topography. FIG. 13 shows the expression of ZO-1 and Na⁺/K⁺-ATPase of HCECs passaged from unpatterned and 1 μm pillar patterned TCPS at P3 and seeded on unpatterned TCPS at P4. As can be seen from the figures, HCECs maintained their phenotype after they have been cultured on patterned TCPS.

It is interesting to note that the cells that were previously grown on 1 um pillars demonstrated better profile of tight junction protein ZO-1 as it was mostly located on cell-cell boundaries.

While for the cells that were previously grown on unpatterned TCPS, the expression of ZO-1 was dispersed in the cells which probably lead us to think that the cells were unable to form compact monolayer, a finding which may be very important for designing the surface of HCEC cell sheet carrier. As far the expression of Na⁺/K⁺-ATPase pumps is considered, the cells showed similar expression of this marker on all samples.

The cell density of the confluent cell sheet was also counted and it was observed that the cell density was around 950±50 cells/mm² on all samples which means that the cells are able to form monolayer with enough cells per mm² area to be called it as a functional monolayer as mentioned previously. The HCEC monolayer cell density can further be increased by seeding cells at higher densities.

### REFERENCES

1. Jumblatt M M, Maurice D M, McCulley J P. Transplantation of tissue-cultured corneal endothelium. Invest Ophthalmol Vis Sci 1978;17:1135.

2. Smolin and Thoft's The Cornea Scientific Foundations and Clinical Pract. Philadelphia: Lippincott Williams and Wilkins, 2005.

3. Joyce NC. Proliferative capacity of the corneal endothelium. Prog Retin Eye Res 2003;22:359.

4. Koizumi N, Sakamoto Y, Okumura N, Tsuchiya H, Torii R, Cooper L J, Ban Y, Tanioka H, Kinoshita S. Cultivated corneal endothelial transplantation in a primate: possible future clinical application in corneal endothelial regenerative medicine. Cornea 2008;27 Suppl 1:S48.

5. McLaughlin C R, Tsai R J, Latorre M A, Griffith M. Bioengineered corneas for transplantation and in vitro toxicology. Front Biosci 2009;14:3326.

6. Lai J Y, Chen K H, Hsu W M, Hsiue G H, Lee Y H. Bioengineered human corneal endothelium for transplantation. Arch Ophthalmol 2006;124:1441.

7. Hsiue G H, Lai J Y, Chen K H, Hsu W M. A novel strategy for corneal endothelial reconstruction with a bioengineered cell sheet. Transplantation 2006;81:473.

8. Sumide T, Nishida K, Yamato M, Ide T, Hayashida Y, Watanabe K, Yang J, Kohno C, Kikuchi A, Maeda N, Watanabe H, Okano T, Tano Y. Functional human corneal endothelial cell sheets harvested from temperature-responsive culture surfaces. FASEB J 2006;20:392.

9. Barile FA. Validating and troubleshooting ocular in vitro toxicology tests. J Pharmacol Toxicol Methods;61:136.

10. Werner A, Braun M, Reichl S, Kietzmann M. Establishing and functional testing of a canine corneal construct. Vet Ophthalmol 2008;11:280.

11. Lim M J, Hurst R K, Konynenbelt B J, Ubels J L. Cytotoxicity testing of multipurpose contact lens solutions using monolayer and stratified cultures of human corneal epithelial cells. Eye Contact Lens 2009;35:287.

12. Ammar D A, Kahook M Y. The effects of combination glaucoma medications on ocular surface epithelial cells. Adv Ther 2009;26:970.

13. Yoeruek E, Spitzer M S, Saygili O, Tatar O, Biedermann T, Bartz-Schmidt K U, Szurman P. Comparison of in vitro safety profiles of vancomycin and cefuroxime on human corneal endothelial cells for intracameral use. J Cataract Refract Surg 2008;34:2139.

14. Yoeruek E, Spitzer M S, Tatar O, Biedermann T, Grisanti S, Luke M, Bartz-Schmidt K U, Szurman P. Toxic effects of recombinant tissue plasminogen activator on cultured human corneal endothelial cells. Invest Ophthalmol Vis Sci 2008;49:1392.

15. Maia J, Ribeiro M P, Ventura C, Carvalho R A, Correia I J, Gil M H. Ocular injectable formulation assessment for oxidized dextran-based hydrogels. Acta Biomater 2009;5:1948.

16. Abrams G A, Schaus S S, Goodman S L, Nealey P F, Murphy C J. Nanoscale topography of the corneal epithelial basement membrane and Descemet's membrane of the human. Cornea 2000;19:57.

17. Yim E K, Leong K W. Significance of synthetic nanostructures in dictating cellular response. Nanomedicine 2005;1:10.

18. Bettinger C J, Langer R, Borenstein J T. Engineering substrate topography at the micro- and nanoscale to control cell function. Angew Chem Int Ed Engl 2009;48:5406.

19. Teixeira Al, Abrams G A, Bertics P J, Murphy C J, Nealey P F. Epithelial contact guidance on well-defined micro- and nanostructured substrates. J Cell Sci 2003;116:1881.

20. Karuri N W, Porri T J, Albrecht R M, Murphy C J, Nealey P F. Nano- and microscale holes modulate cell-substrate adhesion, cytoskeletal organization, and -betel integrin localization in SV40 human corneal epithelial cells. IEEE Trans Nanobioscience 2006;5:273.

21. Karuri N W, Liliensiek S, Teixeira Al, Abrams G, Campbell S, Nealey P F, Murphy C J.

Biological length scale topography enhances cell-substratum adhesion of human corneal epithelial cells. J Cell Sci 2004;117:3153.

22. Diehl K A, Foley J D, Nealey P F, Murphy C J. Nanoscale topography modulates corneal epithelial cell migration. J Biomed Mater Res A 2005;75:603.

23. Teixeira Al, McKie G A, Foley J D, Bertics P J, Nealey P F, Murphy C J. The effect of environmental factors on the response of human corneal epithelial cells to nanoscale substrate topography. Biomaterials 2006;27:3945.

24. Liliensiek S J, Campbell S, Nealey P F, Murphy C J. The scale of substratum topographic features modulates proliferation of corneal epithelial cells and corneal fibroblasts. J Biomed Mater Res A 2006;79:185.

25. Evans M D, McFarland G A, Taylor S, Walboomers X F. The response of healing corneal epithelium to grooved polymer surfaces. Biomaterials 2005;26:1703.

26. Dalby M J, Riehle M O, Johnstone H, Affrossman S, Curtis A S. In vitro reaction of endothelial cells to polymer demixed nanotopography. Biomaterials 2002;23:2945.

27. Miller D C, Thapa A, Haberstroh K M, Webster T J. Endothelial and vascular smooth muscle cell function on poly(lactic-co-glycolic acid) with nano-structured surface features. Biomaterials 2004;25:53.

28. Yim E K F, Hu W, Pang S W, Leong K W. Significance of feature size of topography on the phenotypic expression of smooth muscle cells. Tissue Engineering 2006;12:1017.

29. Gospodarowicz D, Mescher A L, Birdwell C R. Stimulation of corneal endothelial cell proliferation in vitro by fibroblast and epidermal growth factors. Experimental Eye Research 1977;25:75.

30. Esmann M. ATPase and phosphatase activity of Na+,K+-ATPase: molar and specific activity, protein determination. Methods Enzymol 1988;156:105.

31. Ekblom P, Lonai P, Talts J F. Expression and biological role of laminin-1. Matrix Biol 2003;22:35.

32. Dalby M J, Yarwood S J, Riehle M O, Johnstone H J, Affrossman S, Curtis A S. Increasing fibroblast response to materials using nanotopography: morphological and genetic measurements of cell response to 13-nm-high polymer demixed islands. Exp Cell Res 2002;276:1.

33. Christopherson G T, Song H, Mao H Q. The influence of fiber diameter of electrospun substrates on neural stem cell differentiation and proliferation. Biomaterials 2009;30:556.

34. Folkman J, Moscona A. Role of cell shape in growth control. Nature 1978;273:345.

35. Koo S, Ahn S, Zhang H, Wang J, Yim E. Human Corneal Keratocyte Response to Micro- and Nano-Gratings on Chitosan and PDMS. Cellular and Molecular Bioengineering 2011;4:399.

36. Yim E K, Pang S W, Leong K W. Synthetic nanostructures inducing differentiation of human mesenchymal stem cells into neuronal lineage. Exp Cell Res 2007;313:1820.

37. Assoian R K, Zhu X. Cell anchorage and the cytoskeleton as partners in growth factor dependent cell cycle progression. Curr Opin Cell Biol 1997;9:93.

38. Roovers K, Davey G, Zhu X, Bottazzi M E, Assoian R K. Alpha5beta1 integrin controls cyclin D1 expression by sustaining mitogen-activated protein kinase activity in growth factor-treated cells. Mol Biol Cell 1999;10:3197.

39. MacCallum D K, Lillie J H, Scaletta L J, Occhino J C, Frederick W G, Ledbetter S R. Bovine corneal endothelium in vitro : Elaboration and organization of a basement membrane. Experimental Cell Research 1982;139:1.

40. Doughty M J. Are there geometric determinants of cell area in rabbit and human corneal endothelial cell monolayers? Tissue Cell 1998;30:537.

41. Collin S P, Collin H B. A comparative study of the corneal endothelium in vertebrates. Clinical and Experimental Optometry 1998;81:245.

42. Ide T, Nishida K, Yamato M, Sumide T, Utsumi M, Nozaki T, Kikuchi A, Okano T, Tano Y. Structural characterization of bioengineered human corneal endothelial cell sheets fabricated on temperature-responsive culture dishes. Biomaterials 2006;27:607.

43. Hodson S, Miller F. The bicarbonate ion pump in the endothelium which regulates the hydration of rabbit cornea. J Physiol 1976;263:563.

44. Wigham C G, Guggenheim J A, Hodson S A. Sodium movement into and out of corneal endothelium. Pflugers Arch 1994;428:577.

45. Thiagarajah J R, Verkman A S. Aquaporin deletion in mice reduces corneal water permeability and delays restoration of transparency after swelling. J Biol Chem 2002;277:19139.

46. Blachly-Dyson E, Forte M. VDAC channels. IUBMB Life 2001;52:113.

47. Gonzalez-Gronow M, Kalfa T, Johnson C E, Gawdi G, Pizzo S V. The voltage-dependent anion channel is a receptor for plasminogen kringle 5 on human endothelial cells. J Biol Chem 2003;278:27312.

48. Ahn J I, Lee D H, Ryu Y H, Jang I K, Yoon M Y, Shin Y H, Seo Y K, Yoon H H, Kim J C, Song K Y, Yang E K, Kim K H, Park J K. Reconstruction of rabbit corneal epithelium on lyophilized amniotic membrane using the tilting dynamic culture method. Artif Organs 2007;31:711.

49. Ljubimov A V, Burgeson R E, Butkowski R J, Michael A F, Sun T T, Kenney M C. Human corneal basement membrane heterogeneity: topographical differences in the expression of type IV collagen and laminin isoforms. Lab Invest 1995;72:461.

50. Shin J S, Jang I K, Kim C W, Kim J C. Development and characterization of a rabbit corneal endothelial cell line. Jpn J Ophthalmol 2004;48:454.

51. Hatou S, Yamada M, Akune Y, Mochizuki H, Shiraishi A, Joko T, Nishida T, Tsubota K.

Role of insulin in regulation of Na+-/K+-dependent ATPase activity and pump function in corneal endothelial cells. Invest Ophthalmol Vis Sci;51:3935.

52. Whikehart DR, Angelos P, Montgomery B. Effects of mannitol on cultured corneal endothelial cell Na,K-ATPase activity. Cornea 1995;14:295.

1. A cell culture vessel for use in the proliferation and differentiation of mammalian corneal endothelial cells or mammalian corneal endothelial stem cells comprising a cell culture surface wherein said culture surface comprises a plurality of upwardly projecting pillars from a surface wherein the pillars are substantially spaced equidistant from each other and provides a culture surface to which said endothelial cells or endothelial stem cells have enhanced adherence and proliferate and differentiate to form a monolayer of functional endothelial cells.

2. The cell culture vessel according to claim 1 wherein said cell culture surface is provided on a substrate adapted to fit within said cell culture vessel.

3. The cell culture vessel according to claim 1 wherein said cell culture vessel or substrate comprises thermoplastic or elastic polymers.

4. The cell culture vessel according to claim 3 wherein said thermoplastic or elastic polymers are selected from the group consisting of: polymethylmethacrylate, polydimethylsiloxane, polysterene, polyester or polypropylene.

5. The cell culture vessel according to claim 4 wherein said polymer is polydimethylsiloxane.

6. The cell culture vessel according to claim 1 wherein said pillars have a substantially circular cross-section.

7. The cell culture vessel according to claim 1 wherein said pillars are between 100 nm and 1 μm in height +/−15%.

8. The cell culture vessel according to claim 7 wherein the height of said pillars is selected from the group consisting of: 100 nm, 200 nm, 300 nm, 400 nm, 500 nm, 600 nm, 700 nm, 800 nm, 900 nm, 1 μm or 2 μm.

9. The cell culture vessel according to claim 8 wherein the height of said pillars is selected from the group consisting of: 200 nm, 210 nm, 220 nm, 230 nm, 240 nm, 250 nm, 260 nm, 270 nm, 280 nm, 290 nm or 300 nm.

10. The cell culture vessel according to claim 1 wherein the spacing between said pillars is between about 100 nm and 300 nm +/−15%.

11. The cell culture vessel according to claim 1 wherein the spacing between said pillars is between about 1 μm and 6 μm +/−15%.

12. The cell culture vessel according to claim 1 wherein said cell culture vessel or substrate is adapted by provision of one or more extracellular matrix proteins to facilitate endothelial cell growth and differentiation.

13. The cell culture vessel according to claim 12 wherein said one or more extracellular matrix proteins comprise laminin or a combination of laminin and chondroitin sulphate.

14. The cell culture vessel according to claim 12 wherein said one or more extracellular matrix proteins comprise fibronectin and collagen.

15. The cell culture vessel according to claim 1 wherein said cell culture vessel or substrate is seeded with a cell culture composition comprising mammalian corneal endothelial cells or mammalian corneal endothelial stem cells and includes a cell culture medium.

16. The cell culture vessel according to claim 15 wherein said cell culture medium is a stabilisation medium and comprises: human endothelial SFM, 5% v/v Fetal Bovine Serum and 1% antimycotic.

17. The cell culture vessel according to claim 15 wherein said cell culture medium is a proliferation medium and comprises: Ham's F12/M199, 5% v/v fetal bovine serum, 20 μg/ml ascorbic acid, and a combination of insulin, transferrin, selenium, 1% antimycotic and 10 ng/ml bFGF.

18. The cell culture vessel according to claim 15 wherein said mammalian corneal endothelial cells or mammalian corneal endothelial stem cells are allogenic or autologous.

19. The cell culture vessel according to claim 15 wherein said mammalian corneal endothelial cells or mammalian corneal endothelial stem cells are isolated from a non-human species.

20. A method for the culture of mammalian corneal endothelial cells or corneal endothelial stem cells comprising the steps:
i) providing a cell culture vessel or substrate according to claim 1 and seeding said vessel or substrate with mammalian corneal endothelial cells or corneal endothelial stem cells; and
ii) providing cell culture conditions to allow the proliferation and differentiation of said cells.

21. The method according to claim 20 wherein said mammalian corneal endothelial cells or mammalian corneal endothelial stem cells are allogenic or autologous.

22. The method according to claim 20 wherein said mammalian corneal endothelial cells or mammalian corneal endothelial stem cells are isolated from a human subject.

23. The method according to claim 20 wherein said corneal endothelial cells or corneal endothelial stem cells are seeded at a concentration of at least 2,500 cells/cm².

24. The method according to claim 20 wherein said corneal endothelial cells or corneal endothelial stem cells are seeded at a concentration of between 40,000 cells/cm² and 60,000 cells/cm².

25. A method for repair of damaged or diseased corneal tissue, comprising administration of an effective amount of a cell population obtained by the method according to claim 20 to an individual in need thereof.

26. A method to screen for an agent wherein said agent affects the proliferation, differentiation or function of mammalian corneal endothelial cells or mammalian corneal endothelial stem cells comprising the steps of:
i) providing a cell culture vessel according to claim 1 comprising corneal endothelial cells or corneal endothelial stem cells;
ii) adding at least one agent to be tested; and
iii) monitoring the activity of the agent with respect to the proliferation, differentiation or function of said corneal endothelial cells or corneal endothelial stem cells.

27. The method according to claim 26 wherein said screen is a high throughput screen wherein said agent[s] is contacted with a plurality of cell culture preparations.

28. The method according to claim 26 wherein said cell culture vessel is adapted to co-operate with a fluorimetric plate reader.
